(19) United States
(12) Patent Application Publication
(10) Pub. No.: US 2008/0233174 A1
(43) Pub. Date: Sep. 25, 2008

(54) HIGH DOSE FILM COMPOSITIONS AND METHODS OF PREPARATION

(75) Inventors: Garry L. Myers, Kingsport, TN (US); Bill J. Boone, Johnson City, TN (US); Samuel D. Hilbert, Jonesboro, TN (US)

Correspondence Address: HOFFMANN & BARON, LLP, 6900 JERICHO TURNPIKE, SYOSSET, NY, 11791, US

(73) Assignee: MONOSOL RX, LLC, Portage, IN (US)

(21) Appl. No.: 12/013,989

(22) Filed: Jan. 14, 2008

Related U.S. Application Data
(60) Provisional application No. 60/880,085, filed on Jan. 12, 2007.

(51) Int. Cl. A61K 9/00; A61P 43/00

(52) U.S. Cl. 424/435

(57) ABSTRACT

This invention relates to films incorporating high amounts of pharmaceutical agents and methods for the preparation of the same. Moreover, the invention relates to the film products and methods of their preparation that demonstrate a non-self-aggregating uniform heterogeneity. Desirably, the films disintegrate in water and may be formed by a controlled drying process, or other process that maintains the required uniformity of the film. Desirably, the films contain a pharmaceutical and/or cosmetic active agent with no more than a 10% variance of the active agent pharmaceutical and/or cosmetic active agent per unit area of the film.

### CROSS REFERENCE TO RELATED APPLICATIONS

This application claims the benefit of U.S. Provisional Application No. 60/880,085, filed Jan. 12, 2007, the entire contents of which are incorporated herein by reference.

### FIELD OF THE INVENTION

The invention relates to rapidly dissolving high dosage films and methods of their preparation. The films may also contain an active ingredient that is evenly distributed throughout the film. The even or uniform distribution is achieved by controlling one or more parameters, and particularly the elimination of air pockets prior to and during film formation and the use of a drying process that reduces aggregation or conglomeration of the components in the film as it forms into a solid structure.

### BACKGROUND OF THE RELATED TECHNOLOGY

Active ingredients, such as drugs or pharmaceuticals, may be prepared in a tablet form to allow for accurate and consistent dosing. However, this form of preparing and dispensing medications has many disadvantages including that a large proportion of adjuvants that must be added to obtain a size able to be handled, that a larger medication form requires additional storage space, and that dispensing includes counting the tablets which has a tendency for inaccuracy. In addition, many persons, estimated to be as much as 28% of the population, have difficulty swallowing tablets. While tablets may be broken into smaller pieces or even crushed as a means of overcoming swallowing difficulties, this is not a suitable solution for many tablet or pill forms. For example, crushing or destroying the tablet or pill form to facilitate ingestion, alone or in admixture with food, may also destroy the controlled release properties.

As an alternative to tablets and pills, films may be used to carry active ingredients such as drugs, pharmaceuticals, and the like. However, historically films and the process of making drug delivery systems therefrom have suffered from a number of unfavorable characteristics that have not allowed them to be used in practice.

Films that incorporate a pharmaceutically active ingredient are disclosed in expired U.S. Pat. No. 4,136,145 to Fuchs, et al. (“Fuchs”). These films may be formed into a sheet, dried and then cut into individual doses. The Fuchs disclosure alleges the fabrication of a uniform film, which includes the combination of water-soluble polymers, surfactants, flavors, sweeteners, plasticizers and drugs. These allegedly flexible films are disclosed as being useful for oral, topical or enteral use. Examples of specific uses disclosed by Fuchs include application of the films to mucosal membrane areas of the body, including the mouth, rectal, vaginal, nasal and ear areas.

Examination of films made in accordance with the process disclosed in Fuchs, however, reveals that such films suffer from the aggregation or conglomeration of particles, i.e., self-aggregation, making them inherently non-uniform. This result can be attributed to Fuchs' process parameters, which although not disclosed likely include the use of relatively long drying times, thereby facilitating intermolecular attractive forces, convection forces, air flow and the like to form such agglomeration.

The formation of agglomerates randomly distributes the film components and any active present as well. When large dosages are involved, a small change in the dimensions of the film would lead to a large difference in the amount of active per film. If such films were to include low dosages of active, it is possible that portions of the film may be substantially devoid of any active. Since sheets of film are usually cut into unit doses, certain doses may therefore be devoid of or contain an insufficient amount of active for the recommended treatment. Failure to achieve a high degree of accuracy with respect to the amount of active ingredient in the cut film can be harmful to the patient. For this reason, dosage forms formed by processes such as Fuchs, would not likely meet the stringent standards of governmental or regulatory agencies, such as the U.S. Federal Drug Administration (“FDA”), relating to the variation of active in dosage forms. Currently, as required by various world regulatory authorities, dosage forms may not vary more than 10% in the amount of active present. When applied to dosage units based on films, this virtually mandates that uniformity in the film be present.

The problems of self-aggregation leading to non-uniformity of a film were addressed in U.S. Pat. No. 4,849,246 to Schmidt (“Schmidt”). Schmidt specifically pointed out that the methods disclosed by Fuchs did not provide a uniform film and recognized that that the creation of a non-uniform film necessarily prevents accurate dosing, which as discussed above is especially important in the pharmaceutical area. Schmidt abandoned the idea that a mono-layer film, such as described by Fuchs, may provide an accurate dosage form and instead attempted to solve this problem by forming a multi-layered film. Moreover, his process is a multi-step process that adds expense and complexity and is not practical for commercial use.

Other U.S. patents directly addressed the problems of particle self-aggregation and non-uniformity inherent in conventional film forming techniques. In one attempt to overcome non-uniformity, U.S. Pat. No. 5,629,003 to Horstmann et al. and U.S. Pat. No. 5,948,430 to Zerbe et al. incorporated additional ingredients, i.e. gel formers and polyhydric alcohols respectively, to increase the viscosity of the film prior to drying in an effort to reduce aggregation of the components in the film. These methods have the disadvantage of requiring additional components, which translates to additional cost and manufacturing steps. Furthermore, both methods employ the use of conventional time-consuming drying methods such as a high-temperature air-bath using a drying oven, drying tunnel, vacuum drier, or other such drying equipment. The long length of drying time aids in promoting the aggregation of the active and other adjuvant, notwithstanding the use of viscosity modifiers. Such processes also run the risk of exposing the active, i.e., a drug, or vitamin C, or other components to prolonged exposure to moisture and elevated temperatures, which may render it ineffective or even harmful.

In addition to the concerns associated with degradation of an active during extended exposure to moisture, the conventional drying methods themselves are unable to provide uniform films. The length of heat exposure during conventional processing, often referred to as the “heat history”, and the manner in which such heat is applied, have a direct effect on the formation and morphology of the resultant film product. Uniformity is particularly difficult to achieve via conventional drying methods where a relatively thicker film, which is well-suited for the incorporation of a drug active, is desired. Thicker uniform films are more difficult to achieve because the surfaces of the film and the inner portions of the film do not experience the same external conditions simultaneously during drying. Thus, observation of relatively thick films made from such conventional processing shows a non-uniform structure caused by convection and intermolecular forces and requires greater than 10% moisture to remain flexible. The amount of free moisture can often interfere over time with the drug leading to potency issues and therefore inconsistency in the final product.

Conventional drying methods generally include the use of forced hot air using a drying oven, drying tunnel, and the like. The difficulty in achieving a uniform film is directly related to the rheological properties and the process of water evaporation in the film-forming composition. When the surface of an aqueous polymer solution is contacted with a high temperature air current, such as a film-forming composition passing through a hot air oven, the surface water is immediately evaporated forming a polymer film or skin on the surface. This seals the remainder of the aqueous film-forming composition beneath the surface, forming a barrier through which the remaining water must force itself as it is evaporated in order to achieve a dried film. As the temperature outside the film continues to increase, water vapor pressure builds up under the surface of the film, stretching the surface of the film, and ultimately ripping the film surface open allowing the water vapor to escape. As soon as the water vapor has escaped, the polymer film surface reforms, and this process is repeated, until the film is completely dried. The result of the repeated destruction and reformation of the film surface is observed as a “ripple effect” which produces an uneven, and therefore non-uniform film. Frequently, depending on the polymer, a surface will seal so tightly that the remaining water is difficult to remove, leading to very long drying times, higher temperatures, and higher energy costs.

Other factors, such as mixing techniques, also play a role in the manufacture of a pharmaceutical film suitable for commercialization and regulatory approval. Air can be trapped in the composition during the mixing process or later during the film making process, which can leave voids in the film product as the moisture evaporates during the drying stage. The film frequently collapse around the voids resulting in an uneven film surface and therefore, non-uniformity of the final film product. Uniformity is still affected even if the voids in the film caused by air bubbles do not collapse. This situation also provides a non-uniform film in that the spaces, which are not uniformly distributed, are occupying area that would otherwise be occupied by the film composition. None of the above-mentioned patents either addresses or proposes a solution to the problems caused by air that has been introduced to the film.

Therefore, there is a need for methods and compositions for film products, particularly high dosage film products, which use a minimal number of materials or components, and which provide a substantially non-self-aggregating uniform heterogeneity throughout the area of the films. Desirably, such films are produced through a selection of a polymer or combination of polymers that will provide a desired viscosity, a film-forming process such as reverse roll coating, and a controlled, and desirably rapid, drying process which serves to maintain the uniform distribution of non-self-aggregated components without the necessary addition of gel formers or polyhydric alcohols and the like which appear to be required in the products and for the processes of prior patents, such as the aforementioned Horstmann and Zerbe patents. Desirably, the films will also incorporate compositions and methods of manufacture that substantially reduce or eliminate air in the film, thereby promoting uniformity in the final film product.

Moreover, conventional films often incorporate high amounts of fillers, sweeteners, flavors, and other components, thereby limiting the amount of pharmaceutically active ingredient that can be incorporated into the film. In fact, conventional films, at best, often incorporate pharmaceutically active ingredients in an amount that is only about 30% by weight of the film.

In view of the drug-loading limitations of conventional strips, more than one film strip may have to be administered to a patient to deliver the desired amount of a pharmaceutically active agent. In addition, or in the alternative, a film having larger dimensions than desired may have to be used. The administration of more than one strip to deliver a requisite amount of pharmaceutically active ingredient, however, is inefficient and costly from a manufacturing standpoint. Moreover, strips having larger dimensions are often undesirable from a consumer-acceptability standpoint. Accordingly, there remains a need for films that incorporate high amounts of pharmaceutically active ingredients.

### SUMMARY OF THE INVENTION

In some embodiments of the invention, there is provided film product including:

(a) at least one polymer; and

(b) at least one active,

wherein the active is present in an amount that is at least about 30% by weight of the total film product and more desirably, in an amount that is at least about 56% by weight of the total film product and, even more desirably, in an amount that is at least about 60% by weight of the total film product.

In other embodiments of the invention, there is provided a method of orally administering an active including the steps of:

(a) preparing a film comprising at least one polymer and at least one active; and

(b) introducing said film to the oral cavity of a mammal,

wherein the at least one active is present in an amount that is at least about 30% by weight of the total film and more desirably, in an amount that is at least about 56% by weight of the total film product and, even more desirably, in an amount that is at least about 60% by weight of the total film product.

In other embodiments of the invention, there is provided a method of orally administering an active comprising the steps of:

(a) preparing a film by the steps of:
(i) combining at least one polymer and at least one active;
(ii) forming said material into a film; and
(iii) drying said film; and

(b) introducing said film to the oral cavity of a mammal,

wherein the at least one active is present in an amount that is at least about 30% to by weight of the total film and more desirably, in an amount that is at least about 56% by weight of the total film product and, even more desirably, in an amount that is at least about 60% by weight of the total film product.

In yet other embodiments of the invention, there is provided a method for making a film product including combining at least one polymer and at least one active to form a film product, wherein the at least one active is present in an amount that is at least about 30% by weight of the total film product, and more desirably, in an amount that is at least about 56% by weight of the total film product and, even more desirably, in an amount that is at least about 60% by weight of the total film product.

### BRIEF DESCRIPTION OF THE DRAWINGS

FIG. 1 shows a side view of a package containing a unit dosage film of the present invention.

FIG. 2 shows a top view of two adjacently coupled packages containing individual unit dosage forms of the present invention, separated by a tearable perforation.

FIG. 3 shows a side view of the adjacently coupled packages of FIG. 2 arranged in a stacked configuration.

FIG. 4 shows a perspective view of a dispenser for dispensing the packaged unit dosage forms, dispenser containing the packaged unit dosage forms in a stacked configuration.

FIG. 5 is a schematic view of a roll of coupled unit dose packages of the present invention.

FIG. 6 is a schematic view of an apparatus suitable for preparation of a pre-mix, addition of an active, and subsequent formation of the film.

FIG. 7 is a schematic view of an apparatus suitable for drying the films of the present invention.

### DETAILED DESCRIPTION OF THE INVENTION

### High Dosage Film Compositions or Products and Methods of Making and Using the Same

In some embodiments, the present invention provides high dosage film compositions and products which may include up to at least about 56% by weight of an active such as a pharmaceutical agent and, more desirably, up to at least about 60% by weight of an active such as a pharmaceutical agent. In particular, in some embodiments, by not including a plasticizer (other than a self-plasticizing polymer as defined herein) in the present film compositions and products, it is possible to incorporate up to at least about 56% by weight, and, more desirably, up to at least about 60% by weight of an active such as a pharmaceutical agent in the present inventive films to achieve a high dosage film composition or product. As used herein, the term “high dosage film composition or product” refers to a film composition or product that contains an active, particularly a pharmaceutical agent, that is present in an amount that is at least about 30% by weight of the film composition or product. In some embodiments, the high dosage film compositions or products of the present invention may include up to at least about 56% by weight of an active such as a pharmaceutical agent, and, more desirably, up to at least about 60% by weight of an active such as a pharmaceutical agent, and do not contain a plasticizer which is not a self-plasticizing polymer. Desirably, such high dosage film compositions or products of the present invention contain a maximum of only about 4% by weight of sweeteners and/or flavors and/or cosmetic agents and/or taste-masking agents and/or other optional components as identified herein.

In embodiments where a plasticizer is not used which is not a self-plasticizing polymer, the high dosage film compositions and products are desirably formulated to have an overall property of being self-plasticizing and flexible at room temperature. To impart self-plasticity and flexibility to the high dosage film compositions and products, the polymer system used in the high dosage film compositions and products desirably has an overall property of being self-plasticizing and flexible at room temperature. Thus, the polymers used in the present inventive compositions and products desirably have underlying viscoelastic properties, tensile strength, and a Tg (glass transition temperature) which render the polymers self-plasticizing and flexible at room temperature and which allow high doses of actives such as pharmaceutical agents to be incorporated into the present inventive high dosage film compositions and products. In particular, the polymers which are used in the present inventive compositions and products desirably have a tensile strength which allows the polymers to hold the pharmaceutical agents strongly and a Tg which allows the polymers to be flexible enough so that the polymer has the overall property of being self-plasticizing and flexible at room temperature. When the polymers have the overall property of being self-plasticizing and flexible at room temperature, the high dosage film compositions and film products into which the polymers are incorporated will also have the overall property of being self-plasticizing and flexible without the use of a separate plasticizer or plasticizers. Although the molecular weight of the polymers may play a part in the characteristics of the high dosage film compositions and products into which they are incorporated, it will be also understood that the underlying viscoelastic properties, tensile strength, and Tg are also important in making the polymers self-plasticizing and flexible.

Accordingly, by not using a separate plasticizer or separate plasticizers in the present inventive film compositions and products, it is possible “to save” space in the film compositions, thereby allowing high dosages of actives to be incorporated therein. Thus, it will be understood that the strength (particularly, the tensile strength) and the flexibility (Tg) of the polymers in balance desirably allow for the loading of large amounts of actives into the high dosage film compositions and products by obviating the need for a separate plasticizer. Specifically, the polymers of the present invention are desirably “self-plasticizing,” thereby obviating the need for a separate plasticizer by imparting flexibility to the film compositions and products into which they are incorporated.

By way of background, when non-self-plasticizing polymers are used in film compositions and products, it is often necessary to also use plasticizers in the film compositions and products to make the non-self-plasticizing polymers flexible enough for use in the film compositions and products. In particular, the plasticizers are often used to create more free volume space or distance between different segments of the polymer. This decreases the Tg of non-plasticizing polymers by allowing molecular motion to occur between the different polymer molecules thus making the polymers flexible if enough plasticizer is used. Accordingly, plasticizers are often incorporated in film compositions and products in large amounts (between about 20 to 30% by weight for example of a film composition or product, for example) when non-self-plasticizing polymers are used. However, this large amount of plasticizer takes up space in the films compositions and products that could be used for the active if no plasticizer were used. Thus, in contrast to conventional film compositions which use plasticizers, by eliminating the need for separate plasticizers, the present film compositions and products “save” space for actives, thereby allowing high loading of the film compositions and products with actives. In particular, by eliminating the need for separate plasticizers in favor of self-plasticizing polymers systems, it is possible to incorporate actives in an amount up to at least about 56% by weight of the film compositions and products.

Thus, in some embodiments, it is particularly desirable to use “self-plasticizing polymers” in the present inventive film compositions and products. By “self-plasticizing polymers” is meant polymers that will stay flexible at room temperature without the aid of added plasticizers. In other words, the glass transition temperature (Tg) of the polymer is less than room temperature. By “self-plasticizing polymer system” is meant a system which incorporates at least one self-plasticizing polymer.

It will be understood that the self-plasticizing polymers serve a space-saving function when incorporated into the present inventive film compositions and products. By using self-plasticizing polymers in the present inventive high dosage film compositions and products, it is possible to achieve higher loading of an active, such as a pharmaceutical active, than is possible when self-plasticizing polymers are not used. In particular, by using self-plasticizing polymers, it is possible to incorporate from about 20% to about 30% more of an active and, more specifically, from about 20% to about 30% of a pharmaceutical active, into the inventive film compositions and products. The use of self-plasticizing polymers allows about 20% to about 30% of space in the present inventive film compositions and products to “be saved” for additional components, such as pharmaceutical actives, because no additional plasticizers are required. Thus, in some embodiments, high doses of actives can be loaded if the overall Tg of the polymer system is less than room temperature, and this is without the aid of any plasticizers.

As used herein, the term “Tg” refers to the glass transition temperature of a polymer used in the film compositions and products as measured at any time before or after processing of the polymer. Glass transition temperature (Tg) is generally understood to be the temperature at which an amorphous polymer changes from a glass to a rubbery form when an amorphous polymer is heated. The measured value of Tg will depend on the molecular weight of the polymer, on its thermal history and age, on the measurement method, and on the rate of heating or cooling. See Burfield, D. R., Journal of Chemical Education, 1987, 64, 875; Stevens, M. P. Polymer Chemistry: An Introduction, 3^{rd} Ed., Oxford U. Press, NY, 1999. Tg is thus a thermal property which is characteristic of amorphous and semi-crystalline polymers. More particularly, it represents a transition of the polymer from a “rubbery” or “leathery state” to a “glassy state.” Thus, in simple terms, Tg is the temperature below which a polymer goes from rubbery and flexible to brittle and glass-like in nature and above which the polymer is rubbery and flexible.

Tg represents a number of changes in a polymer. In particular, Tg represents a change in the mechanical behavior of a polymer. Below the Tg, a polymer is stiff, hard, and brittle, and above the Tg, a polymer is pliable, soft, and tough. At the Tg, changes in the elastic modulus occur. Moreover, at the Tg, changes in the mobility of the polymer chains are manifest Polymer chains generally lack long-range translational motion. However, above the Tg, the long-range motion (i.e., the segmental motion) of the polymer chains is increased (e.g., chain bending and bond rotation about the segment ends increases (there is an increase in the kinetic energy of the molecules)). In contrast, below the Tg, the chain mobility is suppressed. Additionally, Tg represents changes in the thermodynamic properties of a polymer. In particular, the heat capacity changes and entropy changes. Tg can vary over a wide range of temperatures (<−100° C. to >100° C.) for various polymers. In particular factors which may affect Tg include polymer structure (including structural rigidity and chain mobility), intermolecular forces (secondary forces of polymer chains), chemical composition, and molecular weight. See “POLYMERS, Structure and Bulk Properties”, by Patrick Meares, D. Van Nostrand Company, London, 1965, Principles of polymerization”, by George Odin, John Wiley and Sons, New York,” 1991; <http://www.psrc.usm.edu/macrog/tg.htm>; Thermal Characterization of Polymeric Materials, edited by Edith A. Turi, Press, 1981.

Any suitable self-plasticizing polymer may be used in the present inventive high dosage film compositions and products. Desirably, the self-plasticizing polymers for use in the present inventive high dosage film compositions and products have a Tg less than room temperature (i.e., 30° C.). A particularly useful self-plasticizing polymer for use in the present inventive high dosage film compositions and products is polyethylene oxide. Polyethylene oxide has a Tg less than 0° C. In particular, polyethylene oxide is a thermoplastic semicrystalline polymer with a melting point ranging from about 60° C. to 75° C. and a glass transition temperature of −67° C. See Odian G, ed. Polyethylene oxide. In: Principles of Polymerization, New York, N.Y.: McGraw Hill; 1970: 535-558; Riande et al., eds. Crystalline and amorphous states in polymers. In: Polymer Viscoelasticity: Stress & Strain in Practice, New York, N.Y.: Marcel Dekker Inc.; 2000. Although it is crystalline it retains a high percentage of amorphous region. It is this non-crystalline amorphous region that imparts the self-plasticizing nature to the polymer. Moreover, other useful polymers having a Tg below about 30° C. for use in the present inventive compositions include, for example, polyvinyl acetate (Tg of 18), polymethacrylate (Tg of 20), the polymeric polyethylene glycols, polypropylene glycol, polyethylene/polypropylene glycol copolymer, polyvinylpyrolindone (PVP), and polyoxyethylene alkyl ethers, and combinations thereof.

When polyethylene oxide is used as the self-plasticizing polymer, the polyethylene oxide desirably has a molecular weight ranging from about 100,000 to about 4,000,000. In particular, polyethylene oxide having a molecular weight of about 200,000, polyethylene oxide having a molecular weight of about 600,000, polyethylene oxide having a molecular weight of about 1,000,000, and polyethylene oxide having a molecular weight of about 4,000,000 are all useful in the present inventive high dosage film compositions and products. The molecular weight of the PEO may also be varied. High molecular weight PEO, such as about 4 million, may be desired to increase the mucoadhesivity of the film. In some embodiments, a self-plasticizing polymer (such as polyethylene oxide having a molecular weight of 100,000-300,000) may be combined with another self-plasticizing polymer (such as polyethylene oxide having a molecular weight of 600,000-900,000).

It is well-known that as flexibility increases and molecular weight decreases, the tensile strength of a film composition will decrease. Thus, in formulating the high dosage film compositions and products of the present invention, it is sometimes desirable to increase the tensile strength of the film in order to hold all the particles of active together in a continuous film structure. Accordingly, in some embodiments of the present invention, it is desirable to incorporate a polymer having a Tg above 30° C. in the present inventive high dose compositions along with a low Tg polymer (i.e., a polymer having a Tg below about 30° C.). In particular, a polymer having a Tg above 30° C. may be included in the present inventive high dosage compositions to impart strength to the film compositions. However, it is will be understood that the overall flexible property of the film is still controlled by the low Tg polymer such that flexibility is retained at room temperature.

A particularly useful polymer having a Tg above 30° C. is hydroxypropylmethylcellulose (HPMC), which has a Tg above 100° C. In particular, the Tg of HPMC has been reported to be between 136 and 145° C. See “Aqualon Brochure PTR-025, 2003”. Thus, in some embodiments, a polymer having a Tg above 100° C. (such as HPMC) is combined with at least one polymer having a Tg below about 30° C. (such as PEO, polyvinyl acetate (Tg of 18), polymethacrylate (Tg of 20), the polymeric polyethylene glycols, polypropylene glycol, polyethylene/polypropylene glycol copolymer, polyvinylpyrolindone (PVP), and polyoxyethylene alkyl ethers, and/or combinations thereof. Thus, it will be appreciated that any combination of polymers having a low Tg (i.e., a Tg below about 30° C.) and high Tg (i.e., a Tg above about 30° C.) may be used in the present inventive high dosage film compositions and products.

When a combination of at least one polymer having a Tg below 30° C. and at least one polymer having a Tg above 30° C. is used, it is useful to incorporate the at least one polymer having a Tg below 30° C. in an amount from about 20 to about 40% by weight of the high dosage film composition or product while the at least one polymer having a Tg above 30° C. is desirably incorporated in an amount from about 0.5 to about 10% by weight of the high dosage film composition or product. Desirably, in some embodiments, the molecular weight of the polymer is high (such that the polymer holds the drug particles more strongly) and naturally flexible due to its Tg.

By incorporating at least one polymer having a Tg less than about 30° C. and at least one polymer having a Tg above about 30° C., the resultant high dosage compositions and products will desirably achieve a balance between the properties attributable to the use of both types of polymers. In particular, the present inventive compositions and products may be “high-loaded” with a pharmaceutical active and will desirably exhibit quick dissolution while also exhibiting high tensile strength due to the incorporation of the at least one high molecular polymer. As used herein, compositions and products that are capable of high loading are compositions and products that may contain at least up to about 56% by weight of a pharmaceutical agent. Desirably, the polymer having a Tg less than about 30° C. is a self-plasticizing polymer.

In some embodiments, the self-plasticizing polymer is the same as the active. A particularly useful self-plasticizing polymer which also many be the active is simethicone. Simethicone has such a low Tg that it is liquid at room temperature. In some embodiments, simethicone may be combined with a high Tg polymer, i.e., a polymer having a Tg above about 30° C. (such as hydroxypropylmethyl cellulose).

In some embodiments, by using a pharmaceutical agent having no discernible taste or a taste-masked pharmaceutical agent, it is possible to incorporate at least up to about 60% by weight of a pharmaceutical agent in the present inventive films to achieve a high dosage film composition or product as it will not be necessary to load high amounts of sweeteners and/or flavors and/or cosmetic agents into the film composition or product. As used herein, the term “high dosage film composition or product” refers to a film composition or product that contains a pharmaceutical agent that is present in an amount that is at least about 30% by weight of the film composition or product. In some embodiments, high dosage film compositions or products of the present invention can include at least about 60% by weight of a pharmaceutical agent. Desirably, such high dosage film compositions or products of the present invention contain a maximum of only about 4% by weight of sweeteners and/or flavors and/or cosmetic agents and/or taste-masking agents and/or other optional components as identified herein. Moreover, in some embodiments, where a pharmaceutical agent having no discernible taste is used, no sweetener, flavor, cosmetic agent, or taste-masking agent is added to the high dosage film compositions or products. Additionally, in some embodiments, high dosage film compositions or products of the present invention include no more than about 70% by weight of a polymer and, desirably, no more than about 46% by weight of a polymer.

Such high dosage film compositions or products may be made by combining at least one water-soluble polymer such as a self-plasticizing polymer and at least one pharmaceutical agent to form a film product wherein the at least one pharmaceutical agent is present in an amount that is at least about 30% by weight of the total film composition or product and, more desirably, about 60% by weight of the total film composition or product. In particular, such high dosage film compositions or products can be made by combining at least one water-soluble polymer having a Tg below about 30° C. and at least one pharmaceutical agent to form a film product wherein the at least one pharmaceutical agent is present in an amount that is at least about 30% by weight of the total film composition or product and, more desirably, about 60% by weight of the total film composition or product. In some embodiments, at least one sweetener and/or at least one flavor and/or at least one cosmetic agent and/or at least one other optional component as identified herein may be combined with the polymer and the at least one pharmaceutical agent to form a film composition or product containing no more than about 4% by weight of the at least one sweetener and/or the at least one flavor and/or the least one cosmetic agent and/or the at least one other optional component.

In some embodiments of the invention, there is provided a method of orally administering a pharmaceutical agent that includes preparing a film composition or product by performing the following steps: (i) combining at least one polymer and at least one active such as a pharmaceutical agent; (ii) forming said material into a film; and (iii) drying the film, wherein the at least one active is present in an amount that is at least about 30% by weight of the total film composition or product and, more desirably, wherein the at least one active is present in an amount that is at least about 60% by weight of the total film composition or product. In particular, in some embodiments, there is provided a method of orally administering a pharmaceutical agent that includes the steps of preparing a film composition or product by performing the following steps: (i) combining at least one polymer having a Tg less than 30° C. and at least one pharmaceutical agent; (ii) forming said material into a film; and (iii) drying the film, wherein the at least one pharmaceutical agent is present in an amount that is at least about 30% by weight of the total film composition or product and, more desirably, wherein the at least one pharmaceutical agent is present in an amount that is at least about 60% by weight of the total film composition or product. In yet other embodiments, there is provided a method of orally administering a pharmaceutical agent that includes the steps of preparing a film composition or product by performing the following steps: (i) combining at least one self-plasticizing polymer having a Tg less than 30° C. and at least one pharmaceutical agent; (ii) forming said material into a film; and (iii) drying the film, wherein the at least one pharmaceutical agent is present in an amount that is at least about 30% by weight of the total film composition or product and, more desirably, wherein the at least one pharmaceutical agent is present in an amount that is at least about 60% by weight of the total film composition or product.

After the film composition or product is dried, the film composition or product is introduced into the oral cavity of a mammal. Moreover, in such embodiments, at least one sweetener and/or at least one flavor and/or at least one cosmetic agent and/or at least one other optional component as identified herein may be combined with the water-soluble polymer and the at least one pharmaceutical agent to form a film composition or product containing no more than about 4% by weight of the at least one sweetener and/or the at least one flavor and/or the least one cosmetic agent and/or the at least one other optional component.

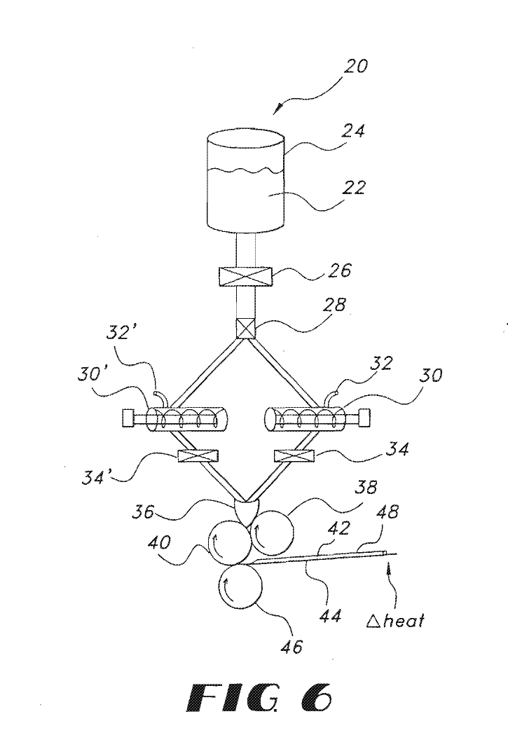

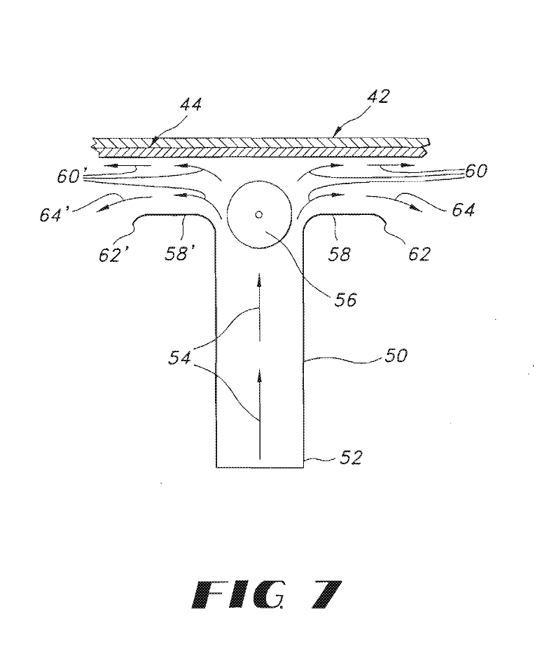

In some embodiments of the invention, the high dosage film compositions and products are prepared by minimizing the amount of time water is in contact with a drug using, for example, mother-daughter mixers. For example, the high dosage film compositions and products of the present invention may be prepared using the apparatus shown in FIG. 6 including daughter mixers 30, 30′ or using any other sequencing or arrangements of mixers, such as series or combination of parallel and series, as discussed below.

In embodiments of this invention employing particulate active agents, whether coated or not, in high dosage film compositions, it is important that the particles not release the active agent during manufacture of the film, yet provide suitable release in the stomach or mouth during dosing, or during dissolution testing. Thus, the particles must reside intact during mixing, coating, film forming, and drying steps, so that the particles remain ready to dissolve in the finished film only in an appropriate environment. Accordingly, manufacturing conditions must be balanced with the composition of the particles to provide stability during manufacture, yet appropriate release of drug. Note that by employing daughter mixers 30 and 30′ (see FIG. 6) in wet casting embodiments of this invention, and not adding active drug to the master batch 22, there is less concern over stability of the particles during possibly extended periods after the master batch is mixed but prior to film forming operations. With the daughter mixers 30 and 30′, the active agent or other ingredients that are incompatible with extended hold times in the master batch can be mixed just prior to the film forming operations with only minimal contact with the liquid ingredients prior to film forming. Even so, the particles should be stable in the liquid film forming ingredients for a sufficient period of time to compensate for the time required to form and dry the film after the film forming ingredients leave the daughter mixers. This time period may be as long as 30 minutes.

In some embodiments, a master batch of a film composition such as a high dosage film composition may be made by mixing a polymer solution in a mother mixer for a suitable amount of time (such as 30-45 minutes) to form a master-batch mixture. A small aliquot of the master-batch mixture is then pumped out into a daughter mixer. Thereafter, an active agent (such as a pharmaceutical active) which may be coated with a taste-masking agent is then incorporated into the daughter mixer. The process is then repeated. By adding the active agent with the taste-masking agent in the daughter mixer, it is possible to minimize the amount of exposure of the taste-masking agent and drug to the water which is present in the polymer solution. This helps to prevent the taste-masking agent from eroding and thus helps to prevent bitterness.

Any suitable mixers known in the art may be used as the mother and daughter mixers. Suitable mixers include, for example, in-line static mixers which mix as pumping occurs through a pipe line. Suitable mixers also include in-line active mixers, which usually use a rotor-stator type of mixing. Moreover, the mother-mixer may be used with as many daughter mixers as desired. It will be understood that any suitable component for use with the present inventive high dosage film compositions may be mixed with the polymer solution in the master-batch in the mother mixer. Suitable residence times are less than 1 hour, desirably less than 45 minutes, and, in some embodiments, about 40 minutes or less. More desirably, the residence time is less than 30 minutes and, even more desirably, the residence time is less than 20 minutes. Even more desirably, the residence time is less than 2 minutes.

Thus, it will be understood that any suitable process made be used to make the high dosage film compositions of the present invention. For example, in some embodiments, there is provided a process of making a high dosage film composition of the present invention which includes the steps of:

(a) forming a masterbatch premix of at least one polymer and water;

(b) deaerating said premix by mixing;

(c) feeding a predetermined amount of said deaerated premix to at least one mixer;

(d) adding an active component to said at least one mixer;

(e) mixing said active component and said predetermined amount of said premix to form a matrix having a uniform distribution of components;

(f) forming a wet film from said matrix;

(g) providing a surface having top and bottom sides;

(h) feeding said film onto said top side of said surface;

(i) rapidly forming a visco-elastic film by applying hot air currents to said bottom side of said surface with substantially no top air flow to prevent air flow migration and intermolecular forces from creating aggregates or conglomerates thereby maintaining the composition uniform distribution of components;

(j) drying said visco-elastic film to form a self-supporting edible film; and

(k) removing said self-supporting film from said surface, wherein the high dosage film composition which is made contains at least about 30% of an active such as a pharmaceutical active and, more desirably, at least about 56% of an active such as a pharmaceutical active, and even more desirably at least about 60% of an active such as a pharmaceutical active and wherein the pharmaceutical active is optionally taste-masked.

Moreover, in other embodiments, there is provided a process for making an ingestible film having a substantially uniform distribution of components and a desired level of a pharmaceutical or biological active component, comprising the steps of:

(a) forming a masterbatch premix of a water-soluble polymer component and water;

(b) feeding a predetermined amount of said premix to at least one mixer;

(c) adding a pharmaceutical or biological active component to said at least one mixer;

(d) mixing said pharmaceutical or biological active component and said predetermined amount of said premix to form a matrix having a uniform distribution of components;

(e) forming a film from said matrix;

(f) providing a conveyor surface having top and bottom sides;

(g) feeding said film onto said top side of said surface; and

(h) drying said film by applying heat to said bottom side of said conveyor surface and exposing said film to a temperature above a degradation temperature of said pharmaceutical or biological active component, wherein said degradation temperature is 70° C. or higher,

wherein said drying step further comprises rapidly forming a visco-elastic film within about the first 4.0 minutes by applying hot air currents to said bottom side of said surface in the absence of hot air currents on the top side of said surface; and

drying said visco-elastic film to form a self-supporting ingestible film,

wherein said pharmaceutical or biological active component is maintained at said desired level, and wherein said desired level is an amount that is at least about 30% by weight of the film and, more desirably, at least about 56% by weight of the film and even more desirably, at least about 60% by weight of the film, and wherein the pharmaceutical or biological active is optionally taste-masked.

Furthermore, in some embodiments, the high dosage films may be made by using any suitable device (such as a widget) which is capable of cutting the films into elongated strips. The elongated strips then may be folded over into one piece and either welded or “slammed” together. Such a process may be beneficial as thickness may sometimes be a limiting factor to making high dosage films.

Low Dosage Film Compositions or Products and Methods of Making and Using the Same from High Dosage Films

In some embodiments, low dosage film compositions or products may be made from the high dosage films of the present invention. In particular, high dosage films containing at least about 30% by weight to about 60% by weight are prepared according to any of the methods described above. The high dosage films are then cut into small pieces (e.g., ⅛″ by ⅛″ pieces) to obtain small pieces of low dosage films. Specifically, such low dosage films desirably contain 2 mg or less of a pharmaceutical active. Moreover, such low dosage films desirably exhibit compositional uniformity in view of the small size and the low drug content.

### Additional Properties of the High Dosage Films

Desirably, the films of the present invention exhibit non-self-aggregating uniform heterogeneity. For the purposes of the present invention the term non-self-aggregating uniform heterogeneity refers to the ability of the films of the present invention, which are formed from one or more components in addition to a polar solvent, to provide a substantially reduced occurrence of, i.e. little or no, aggregation or conglomeration of components within the film as is normally experienced when films are formed by conventional drying methods such as a high-temperature air-bath using a drying oven, drying tunnel, vacuum drier, or other such drying equipment. The term heterogeneity, as used in the present invention, includes films that will incorporate a single component, such as a polymer, as well as combinations of components, such as a polymer and an active. Uniform heterogeneity includes the substantial absence of aggregates or conglomerates as is common in conventional mixing and heat drying methods used to form films.

Furthermore, the films of the present invention have a substantially uniform thickness, which is also not provided by the use of conventional drying methods used for drying water-based polymer systems. The absence of a uniform thickness detrimentally affects uniformity of component distribution throughout the area of a given film.

The film products of the present invention are produced by a combination of a properly selected polymer and a polar solvent, optionally including an active ingredient as well as other fillers known in the art. These films provide a non-self-aggregating uniform heterogeneity of the components within them by utilizing a selected casting or deposition method and a controlled drying process. Examples of controlled drying processes include, but are not limited to, the use of the apparatus disclosed in U.S. Pat. No. 4,631,837 to Magoon (“Magoon”), herein incorporated by reference, as well as hot air impingement across the bottom substrate and bottom heating plates. Another drying technique for obtaining the films of the present invention is controlled radiation drying, in the absence of uncontrolled air currents, such as infrared and radio frequency radiation (i.e. microwaves).

The objective of the drying process is to provide a method of drying the films that avoids complications, such as the noted “rippling” effect, that are associated with conventional drying methods and which initially dry the upper surface of the film, trapping moisture inside. In conventional oven drying methods, as the moisture trapped inside subsequently evaporates, the top surface is altered by being ripped open and then reformed. These complications are avoided by the present invention, and a uniform film is provided by drying the bottom surface of the film first or otherwise preventing the formation of polymer film formation (skin) on the top surface of the film prior to drying the depth of the film. This may be achieved by applying heat to the bottom surface of the film with substantially no top air flow, or alternatively by the introduction of controlled microwaves to evaporate the water or other polar solvent within the film, again with substantially no top air flow. Yet alternatively, drying may be achieved by using balanced fluid flow, such as balanced air flow, where the bottom and top air flows are controlled to provide a uniform films. In such a case, the air flow directed at the top of the film should not create a condition which would cause movement of particles present in the wet film, due to forces generated by the air currents. Additionally, air currents directed at the bottom of the film should desirably be controlled such that the film does not lift up due to forces from the air. Uncontrolled air currents, either above or below the film, can create non-uniformity in the final film products. The humidity level of the area surrounding the top surface may also be appropriately adjusted to prevent premature closure or skinning of the polymer surface.

This manner of drying the films provides several advantages. Among these are the faster drying times and a more uniform surface of the film, as well as uniform distribution of components for any given area in the film. In addition, the faster drying time allows viscosity to quickly build within the film, further encouraging a uniform distribution of components and decrease in aggregation of components in the final film product. Desirably, the drying of the film will occur within about ten minutes or fewer, or more desirably within about five minutes or fewer.

The present invention yields exceptionally uniform film products when attention is paid to reducing the aggregation of the compositional components. By avoiding the introduction of and eliminating excessive air in the mixing process, selecting polymers and solvents to provide a controllable viscosity and by drying the film in a rapid manner from the bottom up, such films result.

The products and processes of the present invention rely on the interaction among various steps of the production of the films in order to provide films that substantially reduce the self-aggregation of the components within the films. Specifically, these steps include the particular method used to form the film, making the composition mixture to prevent air bubble inclusions, controlling the viscosity of the film forming composition and the method of drying the film. More particularly, a greater viscosity of components in the mixture is particularly useful when the active is not soluble in the selected polar solvent in order to prevent the active from settling out. However, the viscosity must not be too great as to hinder or prevent the chosen method of casting, which desirably includes reverse roll coating due to its ability to provide a film of substantially consistent thickness.

In addition to the viscosity of the film or film-forming components or matrix, there are other considerations taken into account by the present invention for achieving desirable film uniformity. For example, stable suspensions are achieved which prevent solid (such as drug particles) sedimentation in non-colloidal applications. One approach provided by the present invention is to balance the density of the particulate (ρₚ) and the liquid phase (ρ₁) and increase the viscosity of the liquid phase (μ). For an isolated particle, Stokes law relates the terminal settling velocity (Vo) of a rigid spherical body of radius (r) in a viscous fluid, as follows:

Vₒ=(2gr^{r})(ρₚ−ρ₁)/9μ

At high particle concentrations, however, the local particle concentration will affect the local viscosity and density. The viscosity of the suspension is a strong function of solids volume fraction, and particle-particle and particle-liquid interactions will further hinder settling velocity.

Stokian analyses has shown that the incorporation of a third phase, dispersed air or nitrogen, for example, promotes suspension stability. Further, increasing the number of particles leads to a hindered settling effect based on the solids volume fraction. In dilute particle suspensions, the rate of sedimentation, v, can be expressed as:

v/Vₒ=1/(1+κφ)

where κ=a constant, and φ is the volume fraction of the dispersed phase. More particles suspended in the liquid phase results in decreased velocity. Particle geometry is also an important factor since the particle dimensions will affect particle-particle flow interactions.

Similarly, the viscosity of the suspension is dependent on the volume fraction of dispersed solids. For dilute suspensions of non-interaction spherical particles, an expression for the suspension viscosity can be expressed as:

μ/μₒ=1+2.5φ

where μₒ is the viscosity of the continuous phase and φ is the solids volume fraction. At higher volume fractions, the viscosity of the dispersion can be expressed as

μ/μₒ=1+2.5φ+C₁φ²+C₂φ³+ . . .

where C is a constant.

The viscosity of the liquid phase is critical and is desirably modified by customizing the liquid composition to a viscoelastic non-Newtonian fluid with low yield stress values. This is the equivalent of producing a high viscosity continuous phase at rest. Formation of a viscoelastic or a highly structured fluid phase provides additional resistive forces to particle sedimentation. Further, flocculation or aggregation can be controlled minimizing particle-particle interactions. The net effect would be the preservation of a homogeneous dispersed phase.

The addition of hydrocolloids to the aqueous phase of the suspension increases viscosity, may produce viscoelasticity and can impart stability depending on the type of hydrocolloid, its concentration and the particle composition, geometry, size, and volume fraction. The particle size distribution of the dispersed phase needs to be controlled by selecting the smallest realistic particle size in the high viscosity medium, i.e., <500 μm. The presence of a slight yield stress or elastic body at low shear rates may also induce permanent stability regardless of the apparent viscosity. The critical particle diameter can be calculated from the yield stress values. In the case of isolated spherical particles, the maximum shear stress developed in settling through a medium of given viscosity can be given as

τₘₐₓ=3Vμ/2r

For pseudoplastic fluids, the viscosity in this shear stress regime may well be the zero shear rate viscosity at the Newtonian plateau.

A stable suspension is an important characteristic for the manufacture of a pre-mix composition which is to be fed into the film casting machinery film, as well as the maintenance of this stability in the wet film stage until sufficient drying has occurred to lock-in the particles and matrix into a sufficiently solid form such that uniformity is maintained. For viscoelastic fluid systems, a rheology that yields stable suspensions for an extended time period, such as 24 hours, must be balanced with the requirements of high-speed film casting operations. A desirable property for the films is shear thinning or pseudoplasticity, whereby the viscosity decreases with increasing shear rate. Time dependent shear effects such as thixotropy are also advantageous. Structural recovery and shear thinning behavior are important properties, as is the ability for the film to self-level as it is formed.

The rheology requirements for the inventive compositions and films are quite severe. This is due to the need to produce a stable suspension of particles, for example 30-60 wt %, in a viscoelastic fluid matrix with acceptable viscosity values throughout a broad shear rate range. During mixing, pumping, and film casting, shear rates in the range of 10-10⁵ sec.⁻¹ may be experienced and pseudoplasticity is the preferred embodiment.

In film casting or coating, rheology is also a defining factor with respect to the ability to form films with the desired uniformity. Shear viscosity, extensional viscosity, viscoelasticity, structural recovery will influence the quality of the film. As an illustrative example, the leveling of shear-thinning pseudoplastic fluids has been derived as

α^{(n-1/n)}=αₒ^{(n-1/n)}−((n−1)/(2n−1))(τ/K)^{1/n}(2π/λ)^{(3+n)/n}h^{(2n+1)/n}t

where α is the surface wave amplitude, αₒ is the initial amplitude, λ is the wavelength of the surface roughness, and both “n” and “K” are viscosity power law indices. In this example, leveling behavior is related to viscosity, increasing as n decreases, and decreasing with increasing K.

Desirably, the films or film-forming compositions of the present invention have a very rapid structural recovery, i.e. as the film is formed during processing, it doesn't fall apart or become discontinuous in its structure and compositional uniformity. Such very rapid structural recovery retards particle settling and sedimentation. Moreover, the films or film-forming compositions of the present invention are desirably shear-thinning pseudoplastic fluids. Such fluids with consideration of properties, such as viscosity and elasticity, promote thin film formation and uniformity.

Thus, uniformity in the mixture of components depends upon numerous variables. As described herein, viscosity of the components, the mixing techniques and the rheological properties of the resultant mixed composition and wet-casted film are important aspects of the present invention. Additionally, control of particle size and particle shape are further considerations. Desirably, the size of the particulate a particle size of 150 microns or less, for example 100 microns or less. Moreover, such particles may be spherical, substantially spherical, or non-spherical, such as irregularly shaped particles or ellipsoidally shaped particles. Ellipsoidally shaped particles or ellipsoids are desirable because of their ability to maintain uniformity in the film-forming matrix as they tend to settle to a lesser degree as compared to spherical particles.

A number of techniques may be employed in the mixing stage to prevent bubble inclusions in the final film. To provide a composition mixture with substantially no air bubble formation in the final product, anti-foaming or surface-tension reducing agents are employed. Additionally, the speed of the mixture is desirably controlled to prevent cavitation of the mixture in a manner which pulls air into the mix. Finally, air bubble reduction can further be achieved by allowing the mix to stand for a sufficient time for bubbles to escape prior to drying the film. Desirably, the inventive process first forms a masterbatch of film-forming components without active ingredients such as drug particles or volatile materials such as flavor oils. The actives are added to smaller mixes of the masterbatch just prior to casting. Thus, the masterbatch pre-mix can be allowed to stand for a longer time without concern for instability in drug or other ingredients.

When the matrix is formed including the film-forming polymer and polar solvent in addition to any additives and the active ingredient, this may be done in a number of steps. For example, the ingredients may all be added together or a pre-mix may be prepared. The advantage of a pre-mix is that all ingredients except for the active may be combined in advance, with the active added just prior to formation of the film. This is especially important for actives that may degrade with prolonged exposure to water, air or another polar solvent.

FIG. 6 shows an apparatus 20 suitable for the preparation of a pre-mix, addition of an active and subsequent formation of a film. The pre-mix or master batch 22, which includes the film-forming polymer, polar solvent, and any other additives except a drug active is added to the master batch feed tank 24. The components for pre-mix or master batch 22 are desirably formed in a mixer (not shown) prior to their addition into the master batch feed tank 24. Then a pre-determined amount of the master batch is controllably fed via a first metering pump 26 and control valve 28 to either or both of the first and second mixers, 30, 30′. The present invention, however, is not limited to the use of two mixers, 30, 30′, and any number of mixers may suitably be used. Moreover, the present invention is not limited to any particular sequencing of the mixers 30, 30′, such as parallel sequencing as depicted in FIG. 6, and other sequencing or arrangements of mixers, such as series or combination of parallel and series, may suitably be used. The required amount of the drug or other ingredient, such as a flavor, is added to the desired mixer through an opening, 32, 32′, in each of the mixers, 30, 30′. Desirably, the residence time of the pre-mix or master batch 22 is minimized in the mixers 30, 30′. While complete dispersion of the drug into the pre-mix or master batch 22 is desirable, excessive residence times may result in leaching or dissolving of the drug, especially in the case for a soluble drug. Thus, the mixers 30, 30′ are often smaller, i.e. lower residence times, as compared to the primary mixers (not shown) used in forming the pre-mix or master batch 22.

A suitable residence time in a mixer is about 40 minutes or less. Desirably, the residence time is less than 20 minutes. More desirably, the residence time is less than 2 minutes.

After the drug has been blended with the master batch pre-mix for a sufficient time to provide a uniform matrix, a specific amount of the uniform matrix is then fed to the pan 36 through the second metering pumps, 34, 34′. The metering roller 38 determines the thickness of the film 42 and applies it to the application roller. The film 42 is finally formed on the substrate 44 and carried away via the support roller 46.

Suitable apparatuses, include, for example, those made by JIT Systems.

While the proper viscosity uniformity in mixture and stable suspension of particles, and casting method are important in the initial steps of forming the composition and film to promote uniformity, the method of drying the wet film is also important. Although these parameters and properties assist uniformity initially, a controlled rapid drying process ensures that the uniformity will be maintained until the film is dry.

The wet film is then dried using controlled bottom drying or controlled microwave drying, desirably in the absence of external air currents or heat on the top (exposed) surface of the film 48 as described herein. Controlled bottom drying or controlled microwave drying advantageously allows for vapor release from the film without the disadvantages of the prior art. Conventional convection air drying from the top is not employed because it initiates drying at the top uppermost portion of the film, thereby forming a barrier against fluid flow, such as the evaporative vapors, and thermal flow, such as the thermal energy for drying. Such dried upper portions serve as a barrier to further vapor release as the portions beneath are dried, which results in non-uniform films. As previously mentioned some top air flow can be used to aid the drying of the films of the present invention, but it must not create a condition that would cause particle movement or a tippling effect in the film, both of which would result in non-uniformity. If top air is employed, it is balanced with the bottom air drying to avoid non-uniformity and prevent film lift-up on the carrier belt. A balance of top and bottom air flow may be suitable where the bottom air flow functions as the major source of drying and the top air flow is the minor source of drying. The advantage of some top air flow is to move the exiting vapors away from the film thereby aiding in the overall drying process. The use of any top air flow or top drying, however, must be balanced by a number of factors including, but not limited, to rheological properties of the composition and mechanical aspects of the processing. Any top fluid flow, such as air, also must not overcome the inherent viscosity of the film-forming composition. In other words, the top air flow cannot break, distort or otherwise physically disturb the surface of the composition. Moreover, air velocities are desirably below the yield values of the film, i.e., below any force level that can move the liquids in the film-forming compositions. For thin or low viscosity compositions, low air velocity must be used. For thick or high viscosity compositions, higher air velocities may be used. Furthermore, air velocities are desirably low so as to avoid any lifting or other movement of the film formed from the compositions.

Moreover, the films of the present invention may contain particles that are sensitive to temperature, such as flavors, which may be volatile, or drugs, which may have a low degradation temperature. In such cases, the drying temperature may be decreased while increasing the drying time to adequately dry the uniform films of the present invention. Furthermore, bottom drying also tends to result in a lower internal film temperature as compared to top drying. In bottom drying, the evaporating vapors more readily carry heat away from the film as compared to top drying which lowers the internal film temperature. Such lower internal film temperatures often result in decreased drug degradation and decreased loss of certain volatiles, such as flavors.

Furthermore, particles or particulates may be added to the film-forming composition or matrix after the composition or matrix is cast into a film. For example, particles may be added to the film 42 prior to the drying of the film 42. Particles may be controllably metered to the film and disposed onto the film through a suitable technique, such as through the use of a doctor blade (not shown) which is a device which marginally or softly touches the surface of the film and controllably disposes the particles onto the film surface. Other suitable, but non-limiting, techniques include the use of an additional roller to place the particles on the film surface, spraying the particles onto the film surface, and the like. The particles may be placed on either or both of the opposed film surfaces, i.e., the top and/or bottom film surfaces. Desirably, the particles are securably disposed onto the film, such as being embedded into the film. Moreover, such particles are desirably not fully encased or fully embedded into the film, but remain exposed to the surface of the film, such as in the case where the particles are partially embedded or partially encased.

The particles may be any useful organoleptic agent, cosmetic agent, pharmaceutical agent, or combinations thereof. As used herein, the term “pharmaceutical agent” is used interchangeably with the term “pharmaceutically active agent.” Desirably, the pharmaceutical agent has no discernible taste or is taste-masked. Moreover, the pharmaceutical agent is desirably a controlled-release pharmaceutical agent. Useful organoleptic agents include flavors and sweeteners. Useful cosmetic agents include breath-freshening or decongestant agents, such as menthol, including menthol crystals.

Although the inventive process for making the high dosage film compositions is not limited to any particular apparatus for the above-described desirable drying, one particular useful drying apparatus 50 is depicted in FIG. 7. Drying apparatus 50 is a nozzle arrangement for directing hot fluid, such as but not limited to hot air, towards the bottom of the film 42 which is disposed on substrate 44. Hot air enters the entrance end 52 of the drying apparatus and travels vertically upward, as depicted by vectors 54, towards air deflector 56. The air deflector 56 redirects the air movement to minimize upward force on the film 42. As depicted in FIG. 7, the air is tangentially directed, as indicated by vectors 60 and 60′, as the air passes by air deflector 56 and enters and travels through chamber portions 58 and 58′ of the drying apparatus 50. With the hot air flow being substantially tangential to the film 42, lifting of the film as it is being dried is thereby minimized. While the air deflector 56 is depicted as a roller, other devices and geometries for deflecting air or hot fluid may suitably be used. Furthermore, the exit ends 62 and 62′ of the drying apparatus 50 are flared downwardly. Such downward flaring provides a downward force or downward velocity vector, as indicated by vectors 64 and 64′, which tend to provide a pulling or drag effect of the film 42 to prevent lifting of the film 42. Lifting of the film 42 may not only result in non-uniformity in the film or otherwise, but may also result in non-controlled processing of the film 42 as the film 42 and/or substrate 44 lift away from the processing equipment.

Monitoring and control of the thickness of the film also contributes to the production of a uniform film by providing a film of uniform thickness. The thickness of the film may be monitored with gauges such as Beta Gauges. A gauge may be coupled to another gauge at the end of the drying apparatus, i.e. drying oven or tunnel, to communicate through feedback loops to control and adjust the opening in the coating apparatus, resulting in control of uniform film thickness.

The film products are generally formed by combining a properly selected polymer and polar solvent, as well as any active ingredient or filler as desired. Desirably, the solvent content of the combination is at least about 30% by weight of the total combination.

The matrix formed by this combination is formed into a film, desirably by roll coating, and then dried, desirably by a rapid and controlled drying process to maintain the uniformity of the film, more specifically, a non-self-aggregating uniform heterogeneity. The resulting film will desirably contain less than about 10% by weight solvent, more desirably less than about 8% by weight solvent, even more desirably less than about 6% by weight solvent and most desirably less than about 2%. The solvent may be water, a polar organic solvent including, but not limited to, ethanol, isopropanol, acetone, methylene chloride, or any combination thereof. Desirably, solvent is incorporated in the high dosage film compositions of the present invention in an amount that is less than 10% by weight of the film compositions. More desirably, solvent is incorporated in the high dosage film compositions of the present invention in an amount that is less than 5% by weight of the film compositions. Even more desirably, solvent is incorporated in the high dosage film compositions of the present invention in an amount that is less than 3% by weight of the film compositions. In some embodiments of the subject invention, particularly where a high dosage film composition or product as discussed herein is desired, the solvent content of the aforementioned combination is only about 3% by weight of the total combination.

Consideration of the above discussed parameters, such as but not limited to rheology properties, viscosity, mixing method, casting method and drying method, also impact material selection for the different components of the present invention. Furthermore, such consideration with proper material selection provides the compositions of the present invention, including a pharmaceutical and/or cosmetic dosage form or film product having no more than a 10% variance of a pharmaceutical and/or cosmetic active per unit area. In other words, the uniformity of the present invention is determined by the presence of no more than a 10% by weight of pharmaceutical and/or cosmetic variance throughout the matrix. Desirably, the variance is less than 5% by weight, less than 2% by weight, less than 1% by weight, or less than 0.5% by weight.

### Film-Forming Polymers

Any suitable polymer may be included in the present inventive high dosage compositions as long as at least one polymer having a Tg less than about 30° C. is used and is present in an amount sufficient to impart an overall flexibility to the films at room temperature. The polymer may be water soluble, water swellable, water insoluble, or a combination of one or more either water soluble, water swellable or water insoluble polymers. The polymer may include cellulose or a cellulose derivative. Specific examples of useful water-soluble polymers include, but are not limited to, pullulan, hydroxypropylmethyl cellulose, hydroxyethyl cellulose, hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum; arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PEA), poly(lactic acid) (PLA), polydioxanones, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

It is particularly desirable to use a polymer blend of polyethylene oxide (PEO) and polydextrose as a film base in the present inventive film compositions and products, especially in the high dosage film compositions and products discussed herein. In particular, such a polymer blend desirably contains polyethylene oxide and polydextrose in a ratio of from about 80 to about 20.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

A film-forming polymer can be incorporated in the present inventive film compositions and products in any suitable amount. Desirably, where the film composition or product is a high dosage film composition or product as discussed herein, the polymer is present in an amount that is no more than about 70% by weight of the total film composition or product. Most desirably, where the film, composition is a high dosage film composition or product as discussed herein, the polymer is present in an amount that is no more than about 46% by weight of the film composition or product.

### Controlled Release Films

The term “controlled release” is intended to mean the release of active at a pre-selected or desired rate. This rate will vary depending upon the application. Desirable rates include fast or immediate release profiles as well as delayed, sustained or sequential release. Combinations of release patterns, such as initial spiked release followed by lower levels of sustained release of active are contemplated. Pulsed drug releases are also contemplated.

The polymers that are chosen for the high dosage films of the present invention may also be chosen to allow for controlled disintegration of the active. This may be achieved by providing a substantially water insoluble film that incorporates an active that will be released from the film over time. This may be accomplished by incorporating a variety of different soluble or insoluble polymers and may also include biodegradable polymers in combination. Alternatively, coated controlled release active particles may be incorporated into a readily soluble film matrix to achieve the controlled release property of the active inside the digestive system upon consumption.

Films including the high dosage films of the present invention that provide a controlled release of the active are particularly useful for buccal, gingival, sublingual and vaginal applications. The films of the present invention are particularly useful where mucosal membranes or mucosal fluid is present due to their ability to readily wet and adhere to these areas.

The convenience of administering a single dose of a medication which releases active ingredients in a controlled fashion over an extended period of time as opposed to the administration of a number of single doses at regular intervals has long been recognized in the pharmaceutical arts. The advantage to the patient and clinician in having consistent and uniform blood levels of medication over an extended period of time are likewise recognized. The advantages of a variety of sustained release dosage forms are well-known. However, the preparation of a film that provides the controlled release of an active has advantages in addition to those well-known for controlled release tablets. For example, thin films are difficult to inadvertently aspirate and provide an increased patient compliance because they need not be swallowed like a tablet. Moreover, certain embodiments of the inventive films are designed to adhere to the buccal cavity and tongue, where they controllably dissolve. Furthermore, thin films may not be crushed in the manner of controlled release tablets which is a problem leading to abuse of drugs such as Oxycontin.

The actives employed in the present invention may be incorporated into the film compositions of the present invention in a controlled release form. For example, particles of drug may be coated with polymers such as ethyl cellulose or polymethacrylate, commercially available under brand names such as Aquacoat ECD and Eudragit E-100, respectively. Solutions of drug may also be absorbed on such polymer materials and incorporated into the inventive film compositions. Other components such as fats and waxes, as well as sweeteners and/or flavors may also be employed in such controlled release compositions.

The actives may be taste-masked prior to incorporation into the film composition, as set forth in co-pending PCT application titled, Uniform Films For Rapid Dissolve Dosage Form Incorporating Taste-Masking Compositions, (based on U.S. Provisional Application No. Express Mail Label No.: EU552991605 US of the same title, filed Sep. 27, 2003, attorney docket No. 1199-15P) the entire subject matter of which is incorporated by reference herein.

### Actives

When an active is introduced to the film, the amount of active per unit area is determined by the uniform distribution of the film. For example, when the films are cut into individual dosage forms, the amount of the active in the dosage form can be known with a great deal of accuracy. This is achieved because the amount of the active in a given area is substantially identical to the amount of active in an area of the same dimensions in another part of the film. The accuracy in dosage is particularly advantageous when the active is a medicament, i.e. a drug.

The active components that may be incorporated into the films of the present invention include, without limitation, pharmaceutical and cosmetic actives, drugs, medicaments, antigens or allergens such as ragweed pollen, spores, microorganisms, seeds, mouthwash components, flavors, fragrances, enzymes, preservatives, sweetening agents, colorants, spices, vitamins and combinations thereof.

A wide variety of medicaments, bioactive active substances and pharmaceutical compositions may be included in the dosage forms of the present invention. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids. H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs or other preferred active ingredients for use in the present invention include anti-diarrheals such as immodium AD, anti-histamines, anti-tussives, decongestants, vitamins, and breath-fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

Additionally, difenhydramine (19 mg) may be included in the films of the present invention.

An anti-oxidant may also be added to the film to prevent the degradation of an active, especially where the active is photosensitive.

Cosmetic active agents may include breath-freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

In some embodiments, it is possible to produce films including high dosage film products and compositions which will result in an “effervescent explosion”/very pleasant sensation when consumed. In particular, in some embodiments, a powdered effervescent K tablet may be incorporated into vitamin C film strips by using a roller with pressure to firmly embed the powder into the strips. The resulting strip produces an “effervescent explosion”/pleasant taste upon dissolution (which may be less than one second) in the mouth.

Also color additives can be used in preparing the films. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

The films containing flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

When the active is combined with the polymer in the solvent, the type of matrix that is formed depends on the solubilities of the active and the polymer. If the active and/or polymer are soluble in the selected solvent, this may form a solution. However, if the components are not soluble, the matrix may be classified as an emulsion, a colloid, or a suspension.

### Dosages

The film products of the present invention are capable of accommodating a wide range of amounts of the active ingredient. The films are capable of providing an accurate dosage amount (determined by the size of the film and concentration of the active in the original polymer/water combination) regardless of whether the required dosage is high or extremely low. Therefore, depending on the type of active or pharmaceutical composition that is incorporated into the film, the active amount may be as high as about 50 mg, desirably up to about 200 mg or as low as the microgram range, or any amount therebetween.

The film products and methods of the present invention are well-suited for high potency, low dosage drugs. This is accomplished through the high degree of uniformity of the films. Therefore, low dosage drugs, particularly more potent racemic mixtures of actives are desirable.

### Anti-Foaming and De-Foaming Compositions

Anti-foaming and/or de-foaming components may also be used with the films of the present invention. These components aid in the removal of air, such as entrapped air, from the film-forming compositions. As described above, such entrapped air may lead to non-uniform films. Simethicone is one particularly useful anti-foaming and/or de-foaming agent. The present invention, however, is not so limited and other anti-foam and/or de-foaming agents may suitable be used.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polymethylsiloxane and 4-7% silicon dioxide. The mixture is a gray, translucent, viscous fluid which is insoluble in water.

When dispersed in water, simethicone will spread across the surface, forming a thin film of low surface tension. In this way, simethicone reduces the surface tension of bubbles of air located in the solution, such as foam bubbles, causing their collapse. The function of simethicone mimics the dual action of oil and alcohol in water. For example, in an oily solution any trapped air bubbles will ascend to the surface and dissipate more quickly and easily, because an oily liquid has a lighter density compared to a water solution. On the other hand, an alcohol/water mixture is known to lower water density as well as lower the water's surface tension. So, any air bubbles trapped inside this mixture solution will also be easily dissipated. Simethicone solution provides both of these advantages. It lowers the surface energy of any air bubbles that become trapped inside the aqueous solution, as well as lowers the surface tension of the aqueous solution. As the result of this unique functionality, simethicone has an excellent anti-foaming property that can be used for physiological processes (anti-gas in stomach) as well as any for external processes that require the removal of air bubbles from a product.

In order to prevent the formation of air bubbles in the films of the present invention, the mixing step can be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles.

Simethicone may be added to the film-forming mixture as an anti-foaming agent in an amount from about 0.01 weight percent to about 5.0 weight percent, more desirably from about 0.05 weight percent to about 2.5 weight percent, and most desirably from about 0.1 weight percent to about 1.0 weight percent.

### Optional Components

A variety of other components and fillers may also be added to the films of the present invention. These may include, without limitation, surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components. It will be appreciated, however, that although plasticizers (besides the self-plasticizing polymers of the present high dosage film compositions) may be incorporated in the high dosage films and products of the present invention (see Example G herein), the plasticizer will replace either polymer (which will weaken the film) or will replace active (which will lower the amount of active that may be loaded into the film high dosage film compositions and products). Accordingly, as discussed above, it is most desirable to incorporate self-plasticizing polymers into the high dosage film compositions and products of the present invention. Moreover, if polymers having a Tg of greater than about 30° C. at room temperature which desirably function to build tensile strength are used, they are desirably used in combination with a self-plasticizing polymer. Otherwise, an additional plasticizer may be required.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcelluloses esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all components.

Further additives may be inorganic fillers, such as calcium carbonate and the oxides of magnesium aluminum, silicon, titanium, etc. desirably in a concentration range of about 0.02% to about 3% by weight and desirably about 0.02% to about 1% based on the weight of all components.

Where a film composition or product of the present invention is a high dosage film composition or product, such optional components may be included in any suitable amount. Moreover, in some embodiments, a high dosage film composition or product contains no added filler.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of about 0.5% to about 2% by weight of the total composition

It is further useful to add silicon dioxide, calcium silicate, or titanium dioxide in a concentration of about 0.02% to about 1% by weight of the total composition. These compounds act as texturizing agents.

These additives are to be used in amounts sufficient to achieve their intended purpose. Generally, the combination of certain of these additives will alter the overall release profile of the active ingredient and can be used to modify, i.e. impede or accelerate the release.

Lecithin is one surface active agent for use in the present invention. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

As additional modifiers which enhance the procedure and product of the present invention are identified, Applicants intend to include all such additional modifiers within the scope of the invention claimed herein.

Other ingredients include binders which contribute to the ease of formation and general quality of the films. Non-limiting examples of binders include starches, pregelatinized starches, gelatin, polyvinylpyrrolidone, methylcellulose, sodium carboxymethylcellulose, ethylcellulose, polyacrylamides, polyvinyloxoazolidone, and polyvinylalcohols.

### Forming the Film

The films of the present invention must be formed into a sheet prior to drying. After the desired components are combined to form a multi-component matrix, including the polymer, water, and an active or other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or in other words able to maintain their integrity and structure in the absence of a separate support. Furthermore, the films of the present invention may be selected of materials that are edible or ingestible.

Coating or casting methods are particularly useful for the purpose of forming the films of the present invention. Specific examples include reverse roll coating, gravure coating, immersion or dip coating metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termed “Extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

### Drying the Film

The drying step is also a contributing factor with regard to maintaining the uniformity of the film composition. A controlled drying process is particularly important when, in the absence of a viscosity increasing composition or a composition in which the viscosity is controlled, for example by the selection of the polymer, the components within the film may have an increased tendency to aggregate or conglomerate. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se.

When a controlled or rapid drying process is desired, this may be through a variety of methods. A variety of methods may be used including those that require the application of heat. The liquid carriers are removed from the film in a manner such that the uniformity, or more specifically, the non-self-aggregating uniform heterogeneity, that is obtained in the wet film is maintained.

Desirably, the film is dried from the bottom of the film to the top of the film. Desirably, substantially no air flow is present across the top of the film during its initial setting period, during which a solid, visco-elastic structure is formed. This can take place within the first few minutes, e.g. about the first 0.5 to about 4.0 minutes of the drying process. Controlling the drying in this manner, prevents the destruction and reformation of the film's top surface, which results from conventional drying methods. This is accomplished by forming the film and placing it on the top side of a surface having top and bottom sides. Then, heat is initially applied to the bottom side of the film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

The temperature at which the films are dried is about 100° C. or less, desirably about 90° C. or less, and most desirably about 80° C. or less.

Another method of controlling the drying process, which may be used alone or in combination with other controlled methods as disclosed above includes controlling and modifying the humidity within the drying apparatus where the film is being dried. In this manner, the premature drying of the top surface of the film is avoided.

Additionally, it has also been discovered that the length of drying time can be properly controlled, i.e. balanced with the heat sensitivity and volatility of the components, and particularly the flavor oils and drugs. The amount of energy, temperature and length and speed of the conveyor can be balanced to accommodate such actives and to minimize loss, degradation or ineffectiveness in the final film.

A specific example of an appropriate drying method is that disclosed by Magoon. Magoon is specifically directed toward a method of drying fruit pulp. However, the present inventors have adapted this process toward the preparation of thin films.

The method and apparatus of Magoon are based on an interesting property of water. Although water transmits energy by conduction and convection both within and to its surroundings, water only radiates energy within and to water. Therefore, the apparatus of Magoon includes a surface onto which the fruit pulp is placed that is transparent to infrared radiation. The underside of the surface is in contact with a temperature controlled water bath. The water bath temperature is desirably controlled at a temperature slightly below the boiling temperature of water. When the wet fruit pulp is placed on the surface of the apparatus, this creates a “refractance window.” This means that infrared energy is permitted to radiate through the surface only to the area on the surface occupied by the fruit pulp, and only until the fruit pulp is dry. The apparatus of Magoon provides the films of the present invention with an efficient drying time reducing the instance of aggregation of the components of the film.

The films may initially have a thickness of about 500 μm to about 1,500 μm, or about 20 mils to about 60 mils, and when dried have a thickness from about 3 μm to about 250 μm, or about 0.1 mils to about 10 mils. Desirably, the dried films will have a thickness of about 2 mils to about 8 mils, and more desirably, from about 3 mils to about 6 mils. In some embodiments, the thickness of the films may be about 0.012 inch thick with a strip size of approximately ⅞ inch by 1¼ inches. In other embodiments, the thickness of the films may be about 0.015 inch thick with a strip size of approximately ⅞ inch by 1½ inches. In still other embodiments, the film thickness may be 0.005 inches thick with a strip size that is approximately about ⅞ inch by 1½ inches.

In some embodiments, the films of the present invention have a dissolution time of about 3-6 seconds. In other embodiments, the films of the present invention have a dissolution time of about 1-3 seconds.

### Uses of Thin Films

The thin films of the present invention are well-suited for many uses. The high degree of uniformity of the components of the film makes them particularly well-suited for incorporating pharmaceuticals. Furthermore, the polymers used in constriction of the films may be chosen to allow for a range of disintegration times for the films. A variation or extension in the time over which a film will disintegrate may achieve control over the rate that the active is released, which may allow for a sustained release delivery system. In addition, the films may be used for the administration of an active to any of several body surfaces, especially those including mucous membranes, such as oral, anal, vaginal, opthalmological, the surface of a wound, either on a skin surface or within a body such as during surgery, and similar surfaces.

The films may be used to orally administer an active. This is accomplished by preparing the films as described above and introducing them to the oral cavity of a mammal. This film may be prepared and adhered to a second or support layer from which it is removed prior to use, i.e. introduction to the oral cavity. An adhesive may be used to attach the film to the support or backing material which may be any of those known in the art, and is preferably not water soluble. If an adhesive is used, it will desirably be a food grade adhesive that is ingestible and does not alter the properties of the active. Mucoadhesive compositions are particularly useful. The film compositions in many cases serve as mucoadhesive themselves.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the films of the present invention takes advantage of the films' tendency to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

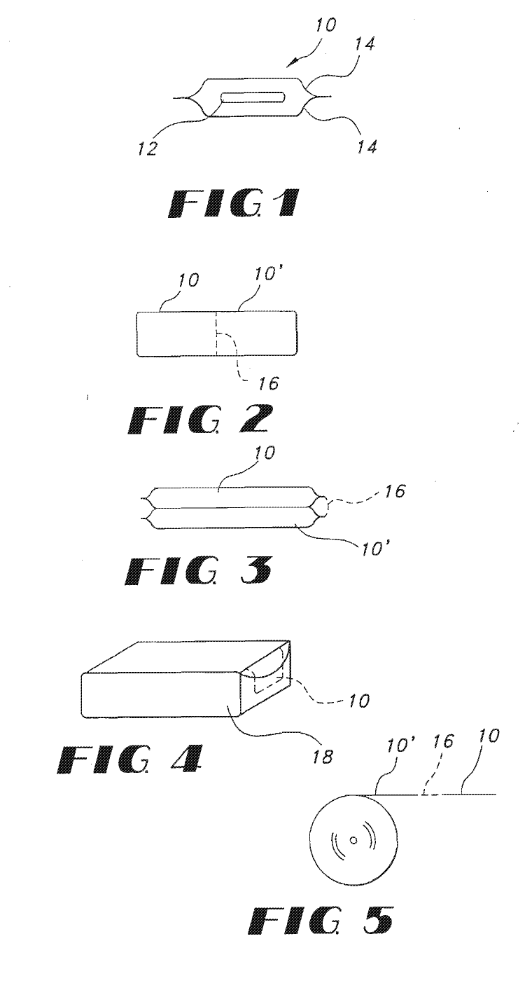

The films of the present invention are desirably packaged in sealed, air and moisture resistant packages to protect the active from exposure oxidation, hydrolysis, volatilization and interaction with the environment. Referring to FIG. 1, a packaged pharmaceutical dosage unit 10, includes each film 12 individually wrapped in a pouch or between foil and/or plastic laminate sheets 14. As depicted in FIG. 2, the pouches 10, 10′ can be linked together with tearable or perforated joints 16. The pouches 10, 10′ may be packaged in a roll as depicted in FIG. 5 or stacked as shown in FIG. 3 and sold in a dispenser 18 as shown in FIG. 4. The dispenser may contain a full supply of the medication typically prescribed for the intended therapy, but due to the thinness of the film and package, is smaller and more convenient than traditional bottles used for tablets, capsules and liquids. Moreover, the films of the present invention dissolve instantly upon contact with saliva or mucosal membrane areas, eliminating the need to wash the dose down with water.

Desirably, a series of such unit doses are packaged together in accordance with the prescribed regimen or treatment, e.g., a 10-90 day supply, depending on the particular therapy. The individual films can be packaged on a backing and peeled off for use.

The features and advantages of the present invention are more fully shown by the following examples which are provided for purposes of illustration, and are not to be construed as limiting the invention in any way.

### EXAMPLES

### Example A

A film cassette containing film strips having the formulation, set forth in Table 1 below was prepared.

TABLE 1

| Ingredient | Approximate % By Weight of Film Strip |
|---|---|
| Film Base¹ | 46% |
| Active Agent² | 50% |
| Other Components³ | 4% |

¹Film base containing a blend of polyethylene oxide (PEO) and polydextrose in a ratio of about 80 to about 20 with added plasticizers.
²Calcium carbonate.
³Flavors, sweeteners, antifoam agents

Each of the strips had weights from about 200 to about 215 mg and contained from about 100 to about 107 mg of active agent depending on the overall weight of the particular strip.

### Example B

A film cassette containing film strips having the formulation set forth in Table 2 below was prepared.

TABLE 2

| Ingredient | Approximate % By Weight of Film Strip |
|---|---|
| Film Base¹ | 46% |
| Active Agent² | 50% |
| Other Components | 4% |

¹Film base containing a blend of polyethylene oxide (PEO) and polydextrose in a ratio of about 80 to about 20 with added plasticizers.
²Calcium carbonate.

Each of the strips had weights from about 290 mg to about 325 mg and contained from about 145 mg to about 162 mg of active agent depending on the overall weight of the particular strip.

### Example C

A film cassette containing film strips having the formulation set forth in Table 3 below was prepared.

TABLE 3

| Ingredient | Approximate % By Weight of Film Strip |
|---|---|
| Film Base¹ | 46% |
| Active Agent² | 50% |
| Other Components | 4% |

¹Blend of polyethylene oxide and polydextrose in a ratio of about 80 to about 20 with added plasticizers.
²Dextromethorphan (not coated).

Each of the strips had weights from about 175 mg to about 195 mg and contained from about 87 mg to about 97 mg of the active agent depending on the overall weight of the particular strip.

### Example D

A film cassette containing film strips having the formulation set forth in Table 4 below was prepared.

TABLE 4

| Ingredient | Approximate % By Weight of Film Strip |
|---|---|
| Film Base¹ | 46% |
| Active Agent² | 50% |
| Other Components | 4% |

¹Blend of polyethylene oxide and polydextrose in a ratio of about 80 to about 20 with added plasticizers.
²Dextromethorphan (not coated).

Each of the strips had weights from about 250 mg to about 275 mg and contained from about 125 mg to about 137 mg of the active agent depending on the overall weight of the particular strip.

### Example E

This example sets forth high dosage films (containing 45 wt. % solids) of the present invention that include a blend of polyethylene oxide (i.e., a self-plasticizing polymer) and polydextrose in a ratio of about 80 to about 20 and at least 50 wt. % of an active agent (particularly, at least 50 wt. % calcium carbonate) as delineated below in Table 5.

TABLE 5

| | | Percent of Total |
|---|---|---|
| Component | Amount in Grams | Composition |
| Polyethylene oxide | 49.68 | 36.8 |
| Polydextrose | 12.42 | 9.2 |
| Precipitated CaCO₃ | 67.5 | 50 |
| Sucralose | 1.35 | 1 |
| Citrus Tango Flavoring | 0.90 | 0.67 |
| Agent | | |
| Vanilla Flavoring Agent | 1.80 | 1.33 |
| Menthol | 1.35 | 1 |
| Distilled Water | 165 | — |
| FD&C Red #40 | 0.034 | approximately 0.025 |
| Coloring Agent | | |
| FD&C Yellow #5 | 0.034 | approximately 0.025 |
| Coloring Agent | | |

The films were prepared by placing the menthol and distilled water in a Degussa 1300 bowl. A blend of polyethylene oxide, polydextrose, calcium carbonate, and sucralose was then added to the bowl. The resultant solution was then stirred in accordance with the conditions set forth below in Table 6 using a Degussa Dental Multivac Compact. After 60 minutes of stirring the FD&C Red #40 and FD&C Yellow #5 coloring agents were added to the mixture. After 64 minutes of stirring, the citrus tango and vanilla flavoring agents were added to the solution.

TABLE 6

| Duration of Stirring | Revolutions Per Minute | |
|---|---|---|
| (minutes) | (rpm) | Vacuum |
| 20 | 150 | 0% |
| 20 | 150 | 50% |
| | | (13 in Hg) |
| 8 | 150 | 75% |
| | | (21.5 in Hg) |
| 12 | 150 | 75% |
| | | (21.5 in Hg) |
| 4 | 150 | 90% |
| | | (24.5 in Hg) |
| 4 | 100 | 100% |
| | | (27 in Hg) |

The solution was then cast into five films using a K-Control Coater with the micrometer adjustable wedge bar set at 300, 600, 900, 1200, and 1500 microns onto 55# PS/1/5 “IN” release paper (available from Griff). The films were dried at 80° C. in accordance with the times set forth below in Table 7. Moreover, the percent moisture of each of the films was determined using a HR73 Moisture Analyzer.

The films were subsequently cut into 1¼ by 1 inch strips, and the strips were weighed. The dosage of calcium carbonate in each strip was then calculated. Moreover, the thickness of each film strip was measured. Additionally, the dissolution rate of the film strips was determined by lowering each film strip into a 36° C. water bath with a 2.85 gram weight and recording the time required for each film strip to separate into two pieces. The results are set forth below in Table 7.

TABLE 7

| | | | | | | | Time required |
|---|---|---|---|---|---|---|---|
| | | | | | Weight of | Dosage CaCO₃ | for dissolution |
| | Micrometer | Drying | | | 1¼ by 1 | per 1¼ by 1 | of 1¼ by 1 |
| | setting on | time | % | Thickness | inch strip | inch strip | inch strip |
| Film | bar | (minutes) | Moisture | (mils) | (mg) | (mg) | (seconds) |
| #1 | 300 | 13 | 1.63 | 3.8 | 100-105 | 50-52.5 | 5 |
| #2 | 600 | 15 | 1.60 | 6.5 | 190-200 | 95-100 | 15 |
| #3 | 900 | 21 | 1.02 | 12 | 300-320 | 150-160 | 40 |
| #4 | 1200 | 34 | 0.74 | 15 | 420-450 | 210-225 | 84 |
| #5 | 1500 | 40 | 0.77 | 17-18 | 520-580 | 260-290 | 93 |

### Example F

This example sets forth high dosage films (containing 45 wt. % solids) of the present invention that include a blend of polyethylene oxide (i.e., a self-plasticizing polymer) and polydextrose (i.e., a filler) for enhancing the dissolution in a ratio of about 60 to about 40 and at least 50 wt. % of an active agent (particularly, at least 50 wt. % of calcium carbonate) as delineated below in Table 8.

TABLE 8

| | Amount | Percent of Total |
|---|---|---|
| Component | in Grams | Composition |
| Polyethylene oxide | 24.81 | 27.57 |
| Polydextrose | 16.54 | 18.08 |
| Precipitated CaCO₃ | 45 | 50 |
| Sucralose | 0.90 | 1 |
| Citrus Tango Flavoring | 0.60 | 0.67 |
| Agent | | |
| Vanilla Flavoring Agent | 1.20 | 1.33 |
| Menthol | 0.90 | 1 |
| Distilled water | 110 | — |
| FD&C Red #40 | 0.022 | 0.025 |
| Coloring Agent | | |
| FD&C Yellow #5 | 0.022 | 0.025 |
| Coloring Agent | | |

The films were prepared by placing FD&C Red #40 coloring agent, FD&C Yellow #5 coloring agent, menthol, and distilled water in a Degussa 1300 bowl. A blend of polyethylene oxide, polydextrose, calcium carbonate, and sucralose was then added to the bowl. The resultant solution was then stirred in accordance with the conditions set forth below in Table 9 using a Degussa Dental Multivac Compact. After 64 minutes of stirring, the citrus tango and vanilla flavoring agents were added to the mixture.

TABLE 9

| Duration of Stirring | Revolutions Per Minute | |
|---|---|---|
| (minutes) | (rpm) | Vacuum |
| 20 | 150 | 0% |
| 20 | 150 | 50% |
| | | (13 in Hg) |
| 20 | 150 | 75% |
| | | (21.5 in Hg) |
| 4 | 150 | 90% |
| | | (24.5 in Hg) |
| 4 | 150 | 100% |
| | | (27 in Hg) |

The solution was then cast into films using a K-Control Coater with the micrometer adjustable wedge bar set at 300, 600, and 900 microns onto 55# PS/1/5 “IN” release paper (available from Griff). The films were dried at 80° C. in accordance with the times set forth below in Table 10. Moreover, the percent moisture of each of the films was determined using a HR73 Moisture Analyzer.

The films were subsequently cut into 1¼ by 1 inch strips, and the strips were weighed. The dosage of calcium carbonate in each strip was then calculated. Moreover, the thickness of each film strip was measured. Additionally, the dissolution rate of the film strips was determined by lowering each film strip into a 36° C. water bath with a 2.85 gram weight and recording the time required for each film strip to separate into two pieces. The results are set forth below in Table 10. As no plasticizer was included in the films, it is not surprising that some film cracking occurred upon removal from the substrate.

TABLE 10

| | | | | | | | Time required |
|---|---|---|---|---|---|---|---|
| | | | | | Weight of | Dosage CaCO₃ | for dissolution |
| | Micrometer | Drying | | | 1¼ by 1 | per 1¼ by 1 | of 1¼ by 1 |
| | setting on | time | % | Thickness | inch strip | inch strip | inch strip |
| Film | bar | (minutes) | Moisture | (mils) | (mg) | (mg) | (seconds) |
| #1 | 300 | 12 | 2.54 | 3.4 | 82-90 | 41-45 | 3 |
| #2 | 600 | 15 | 0.54 | 6.5 | 185-204 | 92.5-100 mg | 16 |
| #3 | 900 | 24 | 0.55 | 11-13 | 310-330 | 155-165 | 36.5 |

### Example G

This example sets forth the properties of high dosage films that include a blend of polyethylene oxide (i.e., a self-plasticizing polymer) and polydextrose in a ratio of about 80 to about 20, at least 50 wt. % of an active agent (particularly, at least 50 wt. % of calcium carbonate), and plasticizers (particularly, propylene glycol and glycerin) as delineated below in Table 11. In particular, this example demonstrates the feasibility of loading higher dosages of drugs in thicker film strips (45 wt. % solids).

TABLE 11

| | | Percent of Total |
|---|---|---|
| Component | Amount in Grams | Composition |
| Polyethylene oxide | 28.13 | 31.25 |
| Polydextrose | 7.03 | 7.81 |
| Precipitated CaCO₃ | 13.5 | 50 |
| Sucralose | 0.90 | 1 |
| Citrus Tango Flavoring | 0.18 | 0.67 |
| Agent | | |
| Vanilla Flavoring Agent | 0.36 | 1.33 |
| Menthol | 0.90 | 1 |
| Distilled water | 110 | — |
| FD&C Red #40 Coloring | 0.022 | 0.025 |
| Agent | | |
| FD&C Yellow #5 Coloring | 0.022 | 0.025 |
| Agent | | |
| Propylene Glycol | 4.14 | 4.6 |
| Glycerin | 2.06 | 2.29 |

The film was prepared by adding the FD&C Red #40 and FD&C Yellow #5 coloring agents, menthol, the propylene glycol, the glycerin, and the distilled water to a Degussa 1300 bowl. A blend of polyethylene oxide, polydextrose, and sucralose was then added to the bowl. The resultant solution was then stirred in accordance with the conditions set forth below in Table 12 below using a Degussa Dental Multivac Compact to form a masterbatch.

TABLE 12

| Duration of Stirring | Revolutions Per Minute | |
|---|---|---|
| (minutes) | (rpm) | Vacuum |
| 20 | 150 | 0% |
| 20 | 150 | 50% |
| | | (13 in Hg) |
| 20 | 150 | 75% |
| | | (21.5 in Hg) |
| 4 | 150 | 90% |
| | | (24.5 in Hg) |
| 4 | 100 | 100% |
| | | (27 in Hg) |

45.966 g of the masterbatch containing 12.962 g solids were then added to a Degussa 1100 bowl. The citrus tango and vanilla flavoring agents were then added to the bowl and stirred in accordance with the conditions set forth in Table 13 below using a Degussa Dental Multivac Compact. After 12 minutes of stirring, the calcium carbonate was added to the mixture.

TABLE 13

| Duration of Stirring | Revolutions Per Minute | |
|---|---|---|
| (minutes) | (rpm) | Vacuum |
| 8 | 150 | 100% |
| | | (27 in Hg) |
| 4 | 100 | 100% |
| | | (27 in Hg) |

The solution was then cast into two films using a K-Control Coater with the micrometer adjustable wedge bar set at 600 and 900 microns onto 55# PS/1/5 “IN” release paper (available from Griff). The films were dried at 80° C. in accordance with the times set forth below in Table 14. Moreover, the percent moisture of each of the films was determined using a HR73 Moisture Analyzer.

The films were subsequently cut into 1¼ by 1 inch strips, and the strips were weighed. The dosage of calcium carbonate in each strip was then calculated. Moreover, the thickness of each film strip was measured. Additionally, the dissolution rate of the film strips was determined by lowering each film strip into a 36° C. water bath with a 2.85 gram weight and recording the time required for each film strip to separate into two pieces. The results are set forth below in Table 14.

TABLE 14

| | | | | | | | Time required |
|---|---|---|---|---|---|---|---|
| | | | | | Weight of | Dosage of CaCO₃ | for dissolution |
| | Micrometer | Drying | | Film | 1¼ by 1 | per 1¼ by 1 | of 1¼ by 1 |
| | setting on | time | % | Thickness | inch strip | inch strip | inch strip |
| Film | bar | (minutes) | Moisture | (mils) | (mg) | (mg) | (seconds) |
| #1 | 600 | 15 | 2.56 | 6.6 | 200-215 | 100-107.5 | 14 |
| #2 | 900 | 22 | 1.42 | 9.6 | 290-325 | 145-162.5 | 33 |

Cassettes of the aforementioned strips were then prepared.

### Example H

This example sets forth the properties of a high dosage film including at least 50 of an active agent (particularly, dextromethorphan (Dx)) as delineated below in Table 15.

45.966 g of the masterbatch prepared as described in Example G were added to a Degussa 1100 bowl. 0.36 g (1.33%) of a vanilla flavoring agent and 0.18 g (0.67%) of a citrus tango flavoring agent were then added to the bowl, and the resultant solution was stirred in accordance with the conditions set forth in Table 15 below using a Degussa Dental Multivac Compact.

TABLE 15

| Duration of Stirring | Revolutions Per Minute | |
|---|---|---|
| (minutes) | (rpm) | Vacuum |
| 8 | 150 | 100% |
| | | (27 in Hg) |
| 4 | 100 | 100% |
| | | (27 in Hg) |

After 12 minutes of stirring 13.5 g (50 wt. %) of coated dextromethorphan, was added to the mixture.

The solution was then cast into two films using a K-Control Coater with the micrometer adjustable wedge bar set at 600 and 900 microns onto 55# PS/1/5 “IN” release paper (available from Griff). The films were dried at 80° C. in accordance with the times set forth below in Table 16. Moreover, the percent moisture of each of the films was determined using a HR73 Moisture Analyzer.

The films were subsequently cut into 1¼ by 1 inch strips, and the strips were weighed. The dosage of dextromethorphan in each strip was then calculated. Moreover, the thickness of each film strip was measured. Additionally, the dissolution rate of the film strips was determined by lowering each film strip into a 36° C. water bath with a 2.85 gram weight and recording the time required for each film strip to separate into two pieces. The results are set forth below in Table 16.

TABLE 16

| | | | | | | | Time required |
|---|---|---|---|---|---|---|---|
| | | | | | Weight of | Dosage of Dx | for dissolution |
| | Micrometer | Drying | | Film | 1¼ by 1 | per 1¼ by 1 | of 1¼ by 1 |
| | setting on | time | % | Thickness | inch strip | inch strip | inch strip |
| Film | bar | (minutes) | Moisture | (mils) | (mg) | (mg)¹ | (seconds) |
| #1 | 600 | 15 | 3.59 | 5.8 | 175-195 | 87.5-97.5 | 19 |
| #2 | 900 | 22 | 2.38 | 19.5 | 250-275 | 125-137.5 | 41 |

¹Assuming 100% w/w mg.

Cassettes of the aforementioned strips were then prepared and packaged.

### Example I

This example sets forth the properties of a high dosage film including at least about 55.85 wt. % of an active agent (particularly, acetaminophen) as delineated below in Table 17. In particular, this example demonstrates the feasibility of incorporating acetaminophen into a film base containing 62.5 wt. % polyethylene oxide (i.e., a self-plasticizing polymer), 6.25 wt. % hydroxypropylmethylcellulose (i.e., a tensile strength builder) 26.56 wt. % starch, and 4.69 wt. % xantural 180 film base (35 wt. % solids) at the 80 mg dose level in a 166.75 mg strip using bubblegum flavor.

TABLE 17

| | | Percent of Total |
|---|---|---|
| Component | Amount in Grams | Composition |
| Polyethylene oxide | 3.50 | 20 |
| Hydroxypropylmethylcellulose | 0.35 | 2 |
| (HPMC E4M) | | |
| Corn Starch | 1.50 | 8.5 |
| Xantural 180 | 0.26 | 1.5 |
| Sucralose | 0.53 | 3 |
| Magna Sweet 100 | 0.087 | 0.5 |
| Microcap acetaminophen | 9.77 | 55.85 |
| Cool Key Flavoring Agent | 0.17 | 1 |
| Bubblegum Flavoring Agent | 1.05 | 6 |
| Butylated Hydroxytoluene | 0.017 | 0.1 |
| FD&C Red #40 Coloring | 0.009 | 0.05 |
| Agent | | |
| Titanium Dioxide | 0.09 | 0.5 |
| Menthol | 0.17 | 1 |
| Distilled water | 32.5 | — |

The film was prepared by adding the FD&C Red #40 coloring agent, the titanium dioxide, the menthol, and the distilled water to a Degussa 1100 bowl. A blend containing the polyethylene oxide, the hydroxypropylmethylcellulose, the corn starch, the xantural 180, the sucralose, and the Magna Sweet 100 was then added to the bowl. The resultant solution was then stirred in accordance with the conditions set forth below in Table 18 below using a Degussa Dental Multivac Compact to form a masterbatch. After 64 minutes of stirring, water was added to compensate for weight loss. Moreover, the Cool Key flavoring agent, the bubblegum flavor, and the butylated hydroxytoluene were added to the solution. After 68 minutes of stirring, the acetaminophen was added to the solution.

TABLE 18

| Duration of Stirring | Revolutions Per Minute | |
|---|---|---|
| (minutes) | (rpm) | Vacuum |
| 20 | 125 | 60% |
| | | (17 in Hg) |
| 20 | 125 | 90% |
| | | (24 in Hg) |
| 12 | 125 | 98% |
| | | (27.5 in Hg) |
| 8 | 125 | 100% |
| | | (28 in Hg) |
| 4 | 125 | 100% |
| | | (28 in Hg) |
| 4 | 100 | 100% |
| | | (28 in Hg) |

The solution was then cast into film using a K-Control Coater with the micrometer adjustable wedge bar set at 780 microns onto the coated side of 6330, which is a high, density polyethylene coated paper which is used as a substrate. The film was dried for 22 minutes in an 85° C. oven. The percent moisture of the film was then determined to be 3.18% using a HR73 Moisture Analyzer.

The film was then cut into 1¼×1 inch strips. Each of the strips weighed between about 155-165 mg.

The film had a film adhesion ratio of 3 from the coated side of 6330. In view of the fact that the film only contained about 22% by weight of polymers (particularly, 20% by weight of polyethylene oxide and 2% by weight of hydroxypropylmethylcellulose), it is not surprising that the film had low tear resistance and a relatively weak strength when pulled. The film, however, passed a 180° bend test when taken out of the moisture analyzer indicating that it is a viable system. Moreover, the film had no particle dragging, had slow to moderate dissolution in the mouth, exhibited no stickiness, had no drug bitterness, did not go to the roof of the mouth, and had good flavor. Although the film had a grainy taste, the film had good flavor.

A cassette of strips was then prepared and packaged.

### Example J

This example sets forth the properties of a high dosage film including at least about 55.85 wt. % of an active agent (particularly, acetaminophen) as delineated below in Table 19. In particular, this example demonstrates the feasibility of incorporating acetaminophen into a film base containing 93.75% polyethylene oxide (molecular weight of 20,000) (i.e., a self-plasticizing polymer) and 6.25% polyethylene oxide (molecular weight of 4,000,000) (i.e., another self-plasticizing polymer) at the 80 mg dose level in a 166.75 mg strip using bubblegum flavor (37.5 wt. % solids reduced to 30 wt. % solids).

TABLE 19

| | Amount | Percent of Total |
|---|---|---|
| Component | in Grams | Composition |
| Polyethylene oxide | 5.63 | 30 |
| (MW = 200,000) | | |
| Polyethylene oxide | 0.37 | 2 |
| (MW = 4,000,000) | | |
| Magna Sweet 100 | 0.094 | 0.5 |
| Sucralose | 0.56 | 3 |
| Microcaps acetaminophen | 10.47 | 55.85 |
| Cool Key Flavoring Agent | 0.19 | 1 |
| Bubblegum Flavoring Agent | 1.12 | 6 |
| Butylated Hydroxytoluene | 0.019 | 0.1 |
| FD&C Red #40 Coloring | 0.009 | 0.05 |
| Agent | | |
| Titanium Dioxide | 0.094 | 0.5 |
| Menthol | 0.19 | 1 |
| Distilled water | 31.25 | — |

The film was prepared by adding the FD&C Red #40 coloring agent, titanium dioxide, menthol, and the distilled water to a Degussa 1110 bowl. A blend containing the polyethylene oxide (molecular weight of 200,000), the polyethylene oxide (molecular weight of 4,000,000), the Magna Sweet 100, and the sucralose was then added to the bowl. The resultant solution was then stirred in accordance with the conditions set forth below in Table 20 below using a Degussa Dental Multivac Compact. The weight of the bowl, stirrer top, and contents prior to stirring was 413.40 grams.

TABLE 20

| Duration of Stirring | Revolutions Per Minute | |
|---|---|---|
| (minutes) | (rpm) | Vacuum |
| 4 | 150 | 60% |
| | | (17 in Hg) |
| 16 | 125 | 60% |
| | | (17 in Hg) |
| 20 | 100 | 90% |
| | | (24 in Hg) |
| 12 | 100 | 98% |
| | | (27.5 in Hg) |
| 8 | 100 | 100% |
| | | (28 in Hg) |
| 4 | 150 | 100% |
| | | (28 in Hg) |
| 4 | 100 | 100% |
| | | (28 in Hg) |

As the weight of the bowl, stirrer top, and contents decreased to 412.30 grams after 60 minutes of stirring, water was added to compensate for water loss. Moreover, the Cool Key flavoring agent, the bubblegum flavoring agent, and the butylated hydroxytoluene were added to the solution after 64 minutes of stirring. Moreover, 3.57 grams of water were then added to yield a mixture containing 35 wt. % solids. After an additional 4 minutes of stirring, the acetaminophen and 8.93 grams of water were added to reduce the content of solids to 30 wt %.

The solution was then cast into film using a K-Control Coater with the micrometer adjustable wedge bar set at 880 microns onto the coated side of 6330. The film was dried for 25 minutes in an 80° C. to 85° C. air oven. The percent moisture of the film was then determined to be 2.60% [using a HR73 Moisture Analyzer.

The film was then cut into 1¼×1 inch strips. Each of the strips weighed about 154 mg. The film had moderate tear resistance and exhibited no particle dragging. In view of the fact that the film only contained about 32% by weight of polymers (particularly, about 30% by weight of polyethylene oxide (MW of 200,000) and about 2% by weight of polyethylene oxide (MW of 4,000,000), it is not surprising that the film had weak strength when pulled. Although the film went slightly to the roof of the mouth, had some grainy taste, and was slightly tacky, the film had no drug bitterness, had good flavor, and has no particle dragging. Moreover, the film had moderate tear resistance and exhibited moderate dissolution in the mouth. The film passed a 180° bend test when taken out of the moisture analyzer.

### Example K

This example sets forth the properties of a high dosage film including at least about 55.85 wt. % of an active agent (particularly, acetaminophen) as delineated below in Table 21. In particular, this example demonstrates the feasibility of incorporating acetaminophen into a film base containing 84.38 wt. % polyethylene oxide (molecular weight of 200,000) (i.e., a self-plasticizing polymer) and 15.62 wt. % polyethylene oxide (molecular weight of 1,000,000) (i.e., another self-plasticizing polymer) at the 80 mg dose level in a 166.75 mg strip using bubblegum flavor (37.5 wt. % solids reduced to 32.5 wt. % solids).

TABLE 21

| | Amount | Percent of Total |
|---|---|---|
| Component | in Grams | Composition |
| Polyethylene oxide | 5.06 | 27 |
| (MW of 200,000) | | |
| Polyethylene oxide | 0.94 | 5 |
| (MW of 1,000,000) | | |
| Magna Sweet 100 | 0.094 | 0.5 |
| Sucralose | 0.56 | 3 |
| Microcap acetaminophen | 10.47 | 55.85 |
| Cool Key Flavoring Agent | 0.19 | 1 |
| Bubblegum Flavoring Agent | 1.12 | 6 |
| Butylated Hydroxytoluene | 0.019 | 0.1 |
| FD&C Red #40 Coloring | 0.009 | 0.05 |
| Agent | | |
| Titanium Dioxide | 0.094 | 0.5 |
| Menthol | 0.19 | 1 |
| Distilled water | 31.25 | — |

The film was prepared by adding the FD&C #40 Red coloring agent, titanium dioxide, menthol, and the distilled water to a Degussa 1100 bowl. A blend containing polyethylene oxide (molecular weight of 200,000), polyethylene oxide (molecular weight of 1,000,000), Magna Sweet 100, and sucralose was then added to the bowl. The weight of the bowl, stirrer top, and contents was 413.57 grams. The resultant solution was then stirred in accordance with the conditions set forth below in Table 22 below using a Degussa Dental Multivac Compact.

TABLE 22

| Duration of Stirring | Revolutions Per Minute | |
|---|---|---|
| (minutes) | (rpm) | Vacuum |
| 4 | 150 | 60% |
| | | (17 in Hg) |
| 16 | 125 | 60% |
| | | (17 in Hg) |
| 20 | 100 | 90% |
| | | (24 in Hg) |
| 12 | 100 | 98% |
| | | (27.5 in Hg) |
| 8 | 100 | 100% |
| | | (28 in Hg) |
| 4 | 125 | 100% |
| | | (28 in Hg) |
| 4 | 100 | 100% |
| | | (28 in Hg) |

As the weight of the bowl, stirrer top, and contents was 412.60 grams after 60 of stirring, water was added to compensate for water loss. Moreover, a solution of the Cook Key flavoring agent, the bubblegum flavoring agent, and the butylated hydroxytoluene was also added after 64 minutes of stirring. After an additional 4 minutes of stirring, the acetaminophen and 7.69 grams of water were added to yield a mixture containing 32.5% solids.

The solution was then cast into film using a K-Control Coater with the micrometer adjustable wedge bar set at 850 microns onto the coated side of 6330 (i.e., high density polyethylene (HDPE)). The film was dried for 25 minutes in an 85° C. air oven. The percent moisture was then determined to be 1.95% using a HR73 Moisture Analyzer.

The film was then cut into 1¼×1 inch strips. Each of the strips weighed between about 158-166 mg.

The film strips had moderate tear resistance, had adequate strength when pulled, had one particle drag, and had slow to moderate dissolution in the mouth. Although the film strips had some grainy taste, the film strips did not go to the roof of the mouth, had no drug bitterness, were not tacky, and had good flavor. The film also passed a 180° bend test when taken out of the moisture analyzer. The HDP side of 6330 had a film release rating of 5 after standing overnight, and the coated side of 6330 came loose on its own. Cassettes of strips were then prepared.

### Example L

This example sets forth the properties of a high dosage film including at least about 55.85 wt. % of an active agent (particularly, acetaminophen) as delineated below in Table 23. In particular, this example demonstrates the feasibility of incorporating acetaminophen into a film base containing 81.253 wt. % polyethylene oxide (molecular weight of 200,000) (i.e., a self-plasticizing polymer) and 18.75 wt. % polyethylene oxide (molecular weight of 600,000) (i.e., another self-plasticizing polymer) at the 80 mg dose level in a 166.75 mg strip using bubblegum flavor (35 wt. % solids reduced to 32.5 wt. % solids).

TABLE 23

| | | Percent of Total |
|---|---|---|
| Component | Amount in Grams | Composition |
| Polyethylene oxide | 4.55 | 26 |
| (MW of 200,000) | | |
| Polyethylene oxide | 1.05 | 6 |
| (MW of 600,000) | | |
| Magna Sweet 100 | 0.09 | 0.5 |
| Sucralose | 0.53 | 3 |
| Microcap acetaminophen | 9.77 | 55.85 |
| Cool Key Flavoring Agent | 0.17 | 1 |
| Bubblegum Flavoring Agent | 1.05 | 6 |
| Butylated Hydroxytoluene | 0.018 | 0.1 |
| FD&C Red #40 Coloring | 0.009 | 0.05 |
| Agent | | |
| Titanium Dioxide | 0.09 | 0.5 |
| Menthol | 0.17 | 1 |
| Distilled water | 32.5 | — |

The film was prepared by adding the FD&C #40 Red coloring agent, titanium dioxide, menthol, and the distilled water to a Degussa 1100 bowl. A blend containing polyethylene oxide (molecular weight of 200,000), polyethylene oxide (molecular weight of 600,000), Magna Sweet 100, and sucralose was then added to the bowl. The weight of the bowl, stirrer top, and contents was 414.37 grams. The resultant solution was then stirred in accordance with the conditions set forth below in Table 24 below using a Degussa Dental Multivac Compact.

TABLE 24

| Duration of Stirring | Revolutions Per Minute | |
|---|---|---|
| (minutes) | (rpm) | Vacuum |
| 4 | 150 | 60% |
| | | (17 in Hg) |
| 16 | 125 | 60% |
| | | (17 in Hg) |
| 20 | 100 | 90% |
| | | (24 in Hg) |
| 12 | 100 | 98% |
| | | (27.5 in Hg) |
| 8 | 100 | 100% |
| | | (28 in Hg) |
| 4 | 125 | 100% |
| | | (28 in Hg) |
| 4 | 100 | 100% |
| | | (28 in Hg) |

As the weight of the bowl, stirrer top, and contents was 413.66 grams after 60 minutes of stirring, water was added to compensate for water loss. Moreover, a solution of the Cool Key flavoring agent, the bubblegum flavoring agent, and the butylated hydroxytoluene also was added after 64 minutes of stirring. After an additional 4 minutes of stirring, the acetaminophen and 3.85 grams of water were added to yield a mixture containing 32.5% solids.

The solution was then cast into film using a K-Control Coater with the micrometer adjustable wedge bar set at 850 microns onto HDP and the coated side of 6330. The film was dried for 25 minutes in an 85° C. air oven. The percent moisture was then determined to be 4.13% using a HR73 Moisture Analyzer.

The film was then cut into 1¼×1 inch strips. Each of the strips weighed between about 157 mg.

The film strips had moderate tear resistance, had adequate strength when pulled, had had no particles dragging, and exhibited slow to moderate dissolution in the mouth. Moreover, although the film strips had a grainy taste, the film strips did not go to the roof of the mouth, had no drug bitterness, were not tacky, and had good flavor. The film also passed a 180° bend test when taken out of the moisture analyzer. The film released initially from the coated side of 5330 but did not release initially from the HDP side of 6330.

### Example M

This example sets forth the properties of a high dosage film including at least about 55.85 wt. % of an active agent (particularly, acetaminophen) as delineated below in Table 25. In particular, this example demonstrates the feasibility of incorporating acetaminophen into a film base containing 0.5 wt. % of Dairy Blend 603-EP (which is a combination of pectin, guar, propylene glycol alginate, and dextrin which functions as a processing aid) at the 80 mg dose level in a 166.75 mg strip using bubblegum flavor (32.5 wt. % solids reduced to 27.5 solids).

TABLE 25

| | | Percent of Total |
|---|---|---|
| Component | Amount in Grams | Composition |
| Polyethylene oxide (MW of | 5.12 | 31.5 |
| 300,000) | | |
| Dairy Blend 603-EP | 0.08 | 0.5 |
| Magna Sweet 100 | 0.08 | 0.5 |
| Sucralose | 6.49 | 3 |
| Microcap acetaminophen | 9.08 | 55.85 |
| Cool Key Flavoring Agent | 0.16 | 1 |
| Bubblegum Flavoring Agent | 0.98 | 6 |
| Butylated Hydroxytolulene | 0.016 | 0.1 |
| FD&C Red #40 | 0.008 | 0.05 |
| Titanium Dioxide | 0.08 | 0.5 |
| Menthol | 0.16 | 1 |
| Distilled water | 33.75 | — |

The film was prepared by adding the FD&C Red #40 coloring agent, titanium dioxide, menthol, and the distilled water to a Degussa 1100 bowl. A blend containing the polyethylene oxide, the dairy blend, the magna sweet 100, and the sucralose was then added to the bowl. The resultant solution was then stirred in accordance with the conditions set forth below in Table 26 below using a Degussa Dental Multivac Compact.

After 20 minutes of stirring, 4.17 grams of water was added to the solution to yield a mixture containing 30 wt. % solids. After 60 minutes of stirring, a solution of the Cool Key flavoring agent, the bubblegum flavoring agent, and the butylated hydroxyltoluene was then added. After an additional 4 minutes of stirring, the acetaminophen and 4.92 grams of water were added to yield a mixture containing 27.5% solids.

TABLE 26

| Duration of Stirring | Revolutions Per Minute | |
|---|---|---|
| (minutes) | (rpm) | Vacuum |
| 8 | 150 | 60% |
| | | (17 in Hg) |
| 4 | 100 | 60% |
| | | (17 in Hg) |
| 8 | 100 | 60% |
| | | (17 in Hg) |
| 20 | 100 | 90% |
| | | (24 in Hg) |
| 12 | 100 | 98% |
| | | (27.5 in Hg) |
| 8 | 100 | 100% |
| | | (28 in Hg) |
| 4 | 100 | 100% |
| | | (28 in Hg) |
| 4 | 100 | 100% |
| | | (28 in Hg) |

The solution was then cast into film using a K-Control Coater with the micrometer adjustable wedge bar set at 980 microns onto the HDP side of 6330 and the coated side of 6330. The film was dried for 28 minutes in an 80° C. air oven. The percent moisture was then determined to be 2.89% using a HR73 Moisture Analyzer.

The film was then cut into 1¼×1 inch strips. Each of the strips weighed between about 167 mg to about 73 mg.

The film had good tear resistance, adequate strength when pulled, had no particle dragging, did not go to the roof of the mouth, and exhibited slow dissolution in the mouth. Although the film had a grainy taste and although the particles adhered together in the mouth to a degree, the film had no drug bitterness and had adequate flavor. The film also passed a 180° bend test when taken out of the moisture analyzer. The film released initially from the coated side of 6330 but would not release initially from the HDP side of 6330. After standing overnight, the film released from the HDP side of 6330 with an adhesion rating of 5.

### Example N

This example sets forth the properties of a high dosage film including at least about 55.85 wt. % of an active agent (particularly, acetaminophen) as delineated below in Table 27. In particular, this example demonstrates the feasibility of incorporating acetaminophen into a film base containing 84.38% of polyethylene oxide (molecular weight of 200,000) (i.e., a self-plasticizing polymer) and 15.62 wt. % of polyethylene oxide (molecular weight of 1,000,000) (i.e., another self-plasticizing polymer) with 3 wt. % starch at the 80 mg dose level in a 166.75 mg strip using bubblegum flavor (32.5 wt. % solids).

TABLE 27

| | | Percent of Total |
|---|---|---|
| Component | Amount in Grams | Composition |
| Polyethylene oxide | 3.98 | 24.47 |
| (molecular weight of | | |
| 200,000) | | |
| Polyethylene oxide | 0.74 | 4.53 |
| (molecular weight of | | |
| 1,000,000) | | |
| Sucralose | 0.49 | 3 |
| Magna Sweet 100 | 0.08 | 0.5 |
| Microcaps acetaminophen | 9.08 | 55.85 |
| Starch | 0.49 | 3 |
| Cool Key Flavoring Agent | 0.16 | 1 |
| Bubblegum Flavoring Agent | 0.97 | 6 |
| Butylated Hydroxytolulene | 0.016 | 0.1 |
| FD&C Red #40 Coloring | 0.008 | 0.05 |
| Agent | | |
| Titanium Dioxide | 0.08 | 0.5 |
| Menthol | 0.16 | 1 |
| Distilled water | 33.75 | — |

The film was prepared by adding the coloring agent, titanium dioxide, menthol, and the distilled water to a Degussa 1100 bowl. A blend containing the polyethylene oxides, the sucralose, and the magna sweet 100 was then added to the bowl. The weight of the bowl, stirrer top, and contents was 414.53 grams. The resultant solution was then stirred in accordance with the conditions set forth in Table 28 below using a Degussa Dental Multivac Compact.

After 60 minutes of stirring, the weight of the bowl, stirrer top, and contents was 413.62 grams. Water was then added to compensate for water loss. Moreover, a solution of the Cook Key flavoring agent, the bubblegum flavoring agent, and the butylated hydroxytoluene was then added. After an additional 4 minutes of stirring, the acetaminophen and starch were then added.

TABLE 28

| Duration of Stirring | Revolutions Per Minute | |
|---|---|---|
| (minutes) | (rpm) | Vacuum |
| 20 | 125 | 60% |
| | | (17 in Hg) |
| 20 | 125 | 90% |
| | | (24 in Hg) |
| 12 | 125 | 98% |
| | | (27.5 in Hg) |
| 8 | 125 | 100% |
| | | (28 in Hg) |
| 4 | 125 | 100% |
| | | (28 in Hg) |
| 4 | 100 | 100% |
| | | (28 in Hg) |

The solution was then cast into film using a K-Control Coater with the micrometer adjustable wedge bar set at 850 microns onto the HDP side of 6330 and the coated side of 6330. The film was dried for 25 minutes in an 80° C. air oven. The percent moisture was then determined to be 3.07% using a HR73 Moisture Analyzer.

The film was then cut into 1¼×1 inch strips. Each of the strips weighed about 162 mg.

The film had moderate tear resistance, had adequate strength when pulled, had no particle dragging, exhibited moderate dissolution in the mouth, and did not go to the roof of the mouth. Although the film had a grainy taste, the film had no drug bitterness and adequate flavor. The film also passed a 180° bend test when taken out of the moisture analyzer. The film released initially from the coated side of 6330 and released from the HDP side of 6330 after standing 5 to 6 hours.

A cassette of strips was then prepared.

### Example O

This example sets forth the properties of a high dosage film including at least about 55.85 wt. % of an active agent (particularly, acetaminophen) as delineated below in Table 29. In particular, this example demonstrates the feasibility of incorporating acetaminophen into a film base containing 84.38% of polyethylene oxide (molecular weight of 200,000) (i.e., a self-plasticizing polymer) and 15.62 wt. % of polyethylene oxide (molecular weight of 1,000,000) (i.e., another self-plasticizing polymer) with 6 wt. % starch at the 80 mg dose level in a 166.75 mg strip using bubblegum flavor (32.5 wt. % solids).

TABLE 29

| | | Percent of Total |
|---|---|---|
| Component | Amount in Grams | Composition |
| Polyethylene oxide | 3.57 | 21.94 |
| 200,000 MW | | |
| Polyethylene oxide | 0.66 | 4.06 |
| 1 million MW | | |
| Sucralose | 0.49 | 3 |
| Magna Sweet 100 | 0.08 | 0.5 |
| Microcaps acetaminophen | 9.08 | 55.85 |
| Starch | 0.97 | 6 |
| Cool Key Flavoring Agent | 0.16 | 1 |
| Bubblegum Flavoring Agent | 0.98 | 6 |
| Butylated Hydroxytoluene | 0.016 | 0.1 |
| FD&C Red #40 Coloring | 0.008 | 0.05 |
| Agent | | |
| Titanium Dioxide | 0.08 | 0.5 |
| Menthol | 0.16 | 1 |
| Distilled water | 33.75 | — |

The film was prepared by adding the coloring agent, titanium dioxide, menthol, and the distilled water to a Degussa 1100 bowl. A blend containing the polyethylene oxides, the sucralose, and the magna sweet 100 was then added to the bowl. The weight of the bowl, stirrer top, and contents was 414.08 grams. The resultant solution was then stirred in accordance with the conditions set forth below in Table 30 using a Degussa Dental Multivac Compact.

After 60 minutes of stirring, the weight of the bowl, stirrer top, and contents was 413.16 grams. Water was then added to compensate for water loss. After 64 minutes of stirring, a solution of the Cool Key flavoring agent, the bubblegum flavoring agent, and the butylated hydroxytoluene was then added. After an additional 4 minutes of stirring, the acetaminophen and starch were then added.

TABLE 30

| Duration of Stirring | Revolutions Per Minute | |
|---|---|---|
| (minutes) | (rpm) | Vacuum |
| 20 | 125 | 60% |
| | | (17 in Hg) |
| 20 | 125 | 90% |
| | | (24 in Hg) |
| 12 | 125 | 98% |
| | | (27.5 in Hg) |
| 8 | 125 | 100% |
| | | (28 in Hg) |
| 4 | 125 | 100% |
| | | (28 in Hg) |
| 4 | 100 | 100% |
| | | (28 in Hg) |

The solution was then cast into film using a K-Control Coater with the micrometer adjustable wedge bar set at 850 microns onto the HDP side of 6330 and the coated side of 6330. The film was dried for 25 minutes in an 80° C. air oven. The percent moisture was then determined to be 2.65% using a HR73 Moisture Analyzer.

The film was then cut into 1¼×1 inch strips. Each of the strips weighed between about 157 mg to about 165 mg.

The film had moderate tear resistance, had adequate strength when pulled, had no particle dragging, exhibited slow to moderate dissolution in the mouth, and did not go to the roof of the mouth. Although the film had a grainy taste, the film had no drug bitterness and adequate flavor. The film also passed a 180° bend test when taken out of the moisture analyzer. The film released initially from the coated side of 6330 and released from the HDP side of 6330 after standing overnight.

### Example P

This example summarizes the film compositions of the present invention.

TABLE 31

| | | % Other Ingredients |
|---|---|---|
| % Polymer | % Active | (Flavor, etc.) |
| 36.80 | 50.00 | 13.20 (Table 5) |
| 27.57 | 50.00 | 22.43 (Table 8) |
| 31.25 | 50.00 | 18.75 (Table 11) |
| 22.00 | 55.85 | 22.15 (Table 17) |
| 32.00 | 55.85 | 12.15 (Table 19) |
| 32.00 | 55.85 | 12.15 (Table 21) |
| 32.00 | 55.85 | 12.15 (Table 23) |
| 32.00 | 55.85 | 12.15 (Table 25) |
| 29.00 | 55.85 | 15.15 (Table 27) |
| 26.00 | 55.85 | 18.15 (Table 29) |
| 12 | 54.52 | 33.48 (Table 32) |

### Example Q

This example sets forth the properties of a high dosage film including at least about 59.52 wt. % of an active agent (particularly, simethicone) as delineated below in Table 32. In particular, this example demonstrates the feasibility of incorporating simethicone into a film base containing 10% of polyethylene oxide (molecular weight of 200,000) (i.e., a self-plasticizing polymer having a low Tg, i.e., a Tg below about 30° C. at room temperature) and hydroxypropylmethyl cellulose (molecular weight of 60,300) (i.e., a polymer which functions as a tensile strength builder and which has a high Tg, i.e., a Tg above about 30° C. at room temperature) with 5.48 wt. % starch in a 105 mg strip peppermint flavor strip (40 wt. % solids). It will be appreciated that the simethicone acts both as a self-plasticizing polymer and as an active.

TABLE 32

| | | Percent of Total |
|---|---|---|
| Component | Amount in Grams | Composition |
| Hydroxypropylmethylcellulose | 9.60 | 12 |
| (HPMC E15) (MW of 60,300; | | |
| viscosity of 15 centipoise) | | |
| Starch | 4.384 | 5.48 |
| Maltrin | 4.384 | 5.48 |
| Polyethylene oxide (MW of | 8.00 | 10 |
| 200,000) | | |
| Fumed Silica¹ | 0.80 | 1 |
| Sucralose | 0.80 | 1 |
| Peppermint Flavor | 1.936 | 2.42 |
| Butylated Hydroxytoluene | 0.08 | 0.1 |
| Blue #1 Coloring Agent | 0.008 | 0.1 |
| Titanium Dioxide | 0.408 | 0.5 |
| Simethicone Formulation² | 49.6 | 62 |
| Distilled Water | 120 | — |

¹Cab-O-Sil available from Cabot.
²Contains 47.616 g (59.52%) simethicone and 1.984 g (248%) other materials.

The film was prepared by adding the coloring agent, 2.48 g (5%) of the simethicone formulation, the titanium dioxide, the menthol, and the distilled water (preheated to 85° C.) to a fabricated glass bowl. A blend containing the hydroxypropylmethylcellulose, the starch, the maltrin, the polyethylene oxide, and the fumed silica was then added to the bowl. The bowl was wrapped with an electric heating tape and the heat was turned on. The solution was prepared as described below using a Degussa Dental Multivac Compact. The weight of the bowl and stirrer top was 1169.88 grams. The resultant solution was then stirred in accordance with the conditions set forth below in Table 33 below using a Degussa Dental Multivac Compact.

TABLE 33

| Duration of Stirring | Heat | Revolutions Per | |
|---|---|---|---|
| (minutes) | (° C.) | Minute (rpm) | Vacuum |
| 12 | 71 | 150 | 0 |

The heat was then cut off and the heating tape was removed. Thereafter, the resultant solution was stirred in accordance with the conditions set forth below in Table 34.

TABLE 34

| Duration of Stirring | Heat | Revolutions Per | |
|---|---|---|---|
| (minutes) | (° C.) | Minute (rpm) | Vacuum |
| 20 | 47 | 200 | 0% |

The sucralose and 47.12 g (95%) of the simethicone formulation was then added to the solution. Thereafter, the resultant solution was stirred in accordance with the conditions set forth in Table 35.

TABLE 35

| Duration of Stirring | Revolutions Per Minute | |
|---|---|---|
| (minutes) | (rpm) | Vacuum |
| 16 | 125 | 60% |
| | | (17 in Hg) |
| 12 | 125 | 90% |
| | | (24 in Hg) |
| 4 | 125 | 95% |
| | | (26.5 in Hg) |
| 8 | 125 | 100% |
| | | (28 in Hg) |

Then, a solution of the peppermint flavor and the butylated hydroxytoluene was added along with 8.30 g of distilled water to compensate for water loss. The resultant solution was then stirred in accordance with the conditions set forth in Table 36.

TABLE 36

| Duration of Stirring | Revolutions Per Minute | |
|---|---|---|
| (minutes) | (rpm) | Vacuum |
| 4 | 125 | 100% |
| | | (28 in Hg) |
| 4 | 100 | 100% |
| | | (28 in Hg) |

A viscosity measurement was then done on the solution using a RVDVE Brookfield Viscometer with Spindle 6 without the guardleg in a mostly filled 4 oz bottle. The viscosity of the solution was 17300 cps (34.6%) at a temperature of 25.2° C.

The solution was then cast into film using a K-Control Coater with the micrometer adjustable wedge bar set at 46° microns onto the HDP side of 6330 and mylar. The film was then dried for 18 minutes in an 80° C. air oven (% moisture=1.69 HR73 Moisture Analyzer). The film was cut into 1.5 by ⅞ inch strips which weighed 107-115 mg. The film had a film adhesion rating of 4 from the HDP side of 6330, had a film adhesion rating of 3-4 from mylar, had 4.8 mil thickness, had moderate dissolution in the mouth, did not go to the roof of the mouth, was not sticky or oily, had no edge creep, had no gummy feel in the mouth, had low to moderate tear resistance, had good strength when pulled, had good flavor, and was borderline on failing the 180° bend test out of the moisturizer analyzer and oven. Strips were then packaged individually. A strip would release from the foil package when opened after being sealed overnight.

While there have been described what are presently believed to be the preferred embodiments of the invention, those skilled in the art will realize that changes and modifications may be made thereto without departing from the spirit of the invention, and it is intended to include all such changes and modifications as fall within the true scope of the invention.

What is claimed is:

1. A film product comprising
(a) at least one polymer; and
(b) at least one active,
wherein the active is present in an amount that is at least about 30% by weight of the total film product.

2. The film product of claim 1, wherein the at least one polymer is a polymer having a Tg less than about 30° C. at room temperature.

3. The film product of claim 2, further comprising at least one polymer having a Tg greater than about 30° C. at room temperature.

4. The film product of claim 1, wherein the at least one polymer is a self-plasticizing polymer.

5. The film product of claim 2, wherein the polymer having a Tg less than about 30° C. is selected from the group consisting of polyethylene oxide, polyvinyl acetate, polymethacrylate, the polymeric polyethylene glycols, polypropylene glycol, polyethylene/polypropylene glycol copolymer, polyvinylpyrolindone, and polyoxyethylene alkyl ethers, and combinations thereof.

6. The film product of claim 3, wherein the polymer having a Tg greater than about 30° C. at room temperature is hydroxypropylmethylcellulose.

7. The film product of claim 2, further comprising at least one second polymer having a Tg less than about 30° C. at room temperature.

8. The film product of claim 7, wherein the at least one second polymer is a polyethylene oxide.

9. The film product of claim 1, wherein the at least one active is present in an amount that is at least about 56% by weight of the film product.

10. The film product of claim 1, wherein the at least one active is present in an amount that is at least about 60% by weight the film product.

11. The film product of claim 1, wherein the polymer is present in an amount of about in an amount from about 20 to about 40% by weight of the film product.

12. The film product of claim 3, wherein the polymer having a Tg greater than about 30° C. at room temperature is present in an amount that is from about 0.5 to about 10% by weight of the film product.

13. The film product of claim 1, wherein the at least one polymer is present in an amount that is no more than about 46% by weight of the total film product.

14. The film product of claim 1, wherein the film product is free of added filler.

15. The film product of claim 1, wherein said film product has a thickness of greater than about 0.1 mils.

16. The film product of claim 1, wherein said film product has a thickness of about 10 mils or fewer.

17. The film product of claim 1, wherein said film product has a substantially uniform thickness.

18. The film product of claim 1, wherein said film product is divided into dosage forms of substantially equal dimensions.

19. The film product of claim 18, wherein each of said dosage forms contains a substantially equal amount of said pharmaceutical agent.

20. The film product of claim 18, wherein said dosage forms contain an amount of said active that varies about 10% or less among said dosage forms.

21. The film product of claim 1, wherein the active has no discernible taste.

22. The film product of claim 1, wherein the active is coated with a taste-masking agent.

23. The film product of claim 1, wherein the active is selected from the group consisting of dextromethorphan, acetaminophen, and simethicone.

24. The film product of claim 1, wherein the film product comprises a filler.

25. The film product of claim 24, wherein the filler is polydextrose.

26. A method of orally administering an active comprising the steps of:
(a) preparing a film comprising at least one polymer and at least one active; and
(b) introducing said film to the oral cavity of a mammal,
wherein the at least one active is present in an amount that is at least about 30% by weight of the total film.

27. The method of claim 26, wherein the at least one polymer is a polymer having a Tg less than about 30° C. at room temperature.

28. The method of claim 26, further comprising at least one polymer having a Tg greater than about 30° at room temperature.

29. The method of claim 26, wherein the at least one polymer is a self-plasticizing polymer.

30. The method of claim 27, wherein the polymer having a Tg less than about 30° C. at room temperature is selected from the group consisting of polyethylene oxide, polyvinyl acetate, polymethacrylate, the polymeric polyethylene glycols, polypropylene glycol, polyethylene/polypropylene glycol copolymer, polyvinylpyrolindone, and polyoxyethylene alkyl ethers, and combinations thereof.

31. The method of claim 28, wherein the at least one polymer having a Tg greater than about 30° C. at room temperature is hydroxypropylmethylcellulose.

32. The method of claim 27, further comprising at least one second polymer having a Tg less than about 30° C. at room temperature.

33. The method of claim 32, wherein the at least one second polymer is a polyethylene oxide.

34. The method of claim 26, wherein the active is present in an amount that is at least about 56% by weight of the film product.

35. The method of claim 26, wherein the active is present in an amount that is at least about 60% by weight the film product.

36. The method of claim 26, wherein the polymer is present in an amount from about 20 to about 40% by weight of the film product.

37. The method of claim 28, wherein the polymer having a Tg greater than about 30° C. is present in an amount that is from about 0.5 to about 10% by weight of the film product.

38. The method of claim 26, wherein the at least one polymer is present in an amount that is no more than about 46% by weight of the total film product.

39. The method of claim 26, wherein the film product is free of added filler.

40. The method of claim 26, wherein said film product has a thickness of greater than about 0.1 mils.

41. The method of claim 26, wherein said film product has a thickness of about 10 mils or fewer.

42. The method of claim 26, wherein said film product has a substantially uniform thickness.

43. The method of claim 26, wherein said film product is divided into dosage forms of substantially equal dimensions.

44. The method of claim 26, wherein each of said dosage forms contains a substantially equal amount of said pharmaceutical agent.

45. The method of claim 26, wherein said dosage forms contain an amount of said active that varies about 10% or less among said dosage forms.

46. The method of claim 26, wherein the active has no discernible taste.

47. The method of claim 26, wherein the active is coated with a taste-masking agent.

48. The method of claim 26, wherein the active is selected from the group consisting of dextromethorphan, acetaminophen, and simethicone.

49. The method of claim 26, wherein the film product comprises a filler.

50. The method of claim 49, wherein the filler is polydextrose.

51. The method of claim 26, wherein the film is prepared by the steps of:
(i) combining the at least one polymer and the at least one active to form a material;
(ii) forming the material into a film; and
(iii) drying the film.

52. A method for making a film product comprising combining at least one polymer and at least one active to form a film product, wherein the at least one active is present in an amount that is at least about 30% by weight of the total film product.

53. The method of claim 52, wherein the at least one active is present in an amount that is at least about 56% by weight of the total film product.

54. The method of claim 52, wherein the active is present in an amount that is at least about 60% by weight of the total film product.
